(19) United States
(12) Patent Application Publication
(10) Pub. No.: US 2014/0179624 A1
(43) Pub. Date: Jun. 26, 2014

(54) CYTOPROTECTIVE DERIVATIVES OF AVICIN D AND METHODS OF MAKING AND USING THEREOF

(71) Applicant: Research Development Foundation, Carson City, NV (US)

(72) Inventors: Jordan GUTTERMAN, Houston, TX (US); Valsala HARIDAS, Pearland, TX (US); Peter C. MICHELS, Voorheesville, NY (US); Yuri L. KHMELNITSKY, Schenectady, NY (US); Vadim M. MOZHAEV, Clifton Park, NY (US)

(73) Assignee: Research Development Foundation, Carson City, NV (US)

(21) Appl. No.: 14/137,449

(22) Filed: Dec. 20, 2013

Related U.S. Application Data
(60) Provisional application No. 61/740,002, filed on Dec. 20, 2012.

(51) Int. Cl. C07H 15/24

(52) U.S. Cl. CPC C07H 15/24
USPC 514/33; 536/18.1

(57) ABSTRACT

Disclosed herein are novel cytoprotective derivatives of avicin D, including those of the formula:

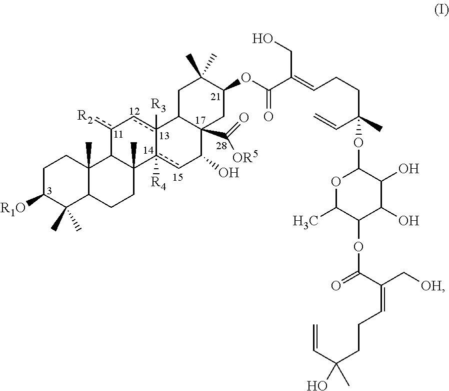

wherein the variables are defined herein. Also provided are pharmaceutical compositions, kits and articles of manufacture comprising these derivative compounds. Methods and intermediates useful for making the derivatives, and methods of using the derivatives and compositions thereof, including for the treatment of cancer, are also provided.

This application claims the benefit of U.S. Provisional Application No. 61/740,002, filed on Dec. 20, 2012, the entirety of which is incorporated herein by reference.

### BACKGROUND OF THE INVENTION

I. Field of the Invention

The present invention relates generally to the fields of biology, chemistry and medicine. More particularly, it concerns derivatives of avicin D, and methods of making and using thereof, including for the treatment of cancer.

II. Description of Related Art

Avicins, a family of plant triterpene electrophiles, have been reported to trigger apoptosis-associated tumor cell death, and suppress chemical-induced carcinogenesis by their anti-inflammatory, anti-mutagenic, and antioxidant properties.

Avicins can be isolated from the Australian desert tree (Leguminosae) Acacia victoriae. The extraction and purification of avicins from the ground pods of Acacia victoriae is described in detail by U.S. Pat. No. 6,444,233 to Arntzen et al., which is incorporated herein by reference. Using induction of cell cytotoxicity as a screen, two compounds, avicin D and avicin G, were identified as having significant activity.

Avicin D has been shown to inhibit NF-κB and activate NF-E2-related factor 2 (Nrf2) respectively, both in a redox-dependant manner, accounting for its anti-inflammatory and antioxidant properties. The ability of avicins to interact with, and modify cysteine residues was first demonstrated in a bacterial system with OxyR as a target, wherein it was demonstrated that the distal portion of the avicin side chain formed a reversible and covalent thioester bond with the critical cysteine (SH) on the OxyR molecule. This protein modification, termed avicinylation, suggested that avicins can be used induce post-translational changes in proteins to regulate their function.

Given these promising properties and the pressing need for improved therepeutics in a diverse range of indications, it is desirable to synthesize new compounds with diverse structures that may have improved biological activity profiles. Therefore, it is an object of the invention to provide derivatives of avicin D, and methods of making and using these.

### SUMMARY OF THE INVENTION

The present disclosure provides novel compounds, including derivatives of Avicin D, methods for their manufacture, and methods for their use, including for the treatment and/or prevention of cancer or other diseases.

In one aspect there are provided compounds of the formula:

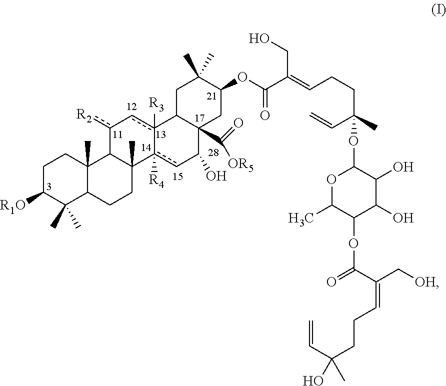

wherein:
R₁ is:
   hydrogen, a monosaccharide group, a disaccharide group, an oligosaccharide group or a terpenoid group, or
   alkyl_{(C≦30)}, alkenyl_{(C≦30)}, alkynyl_{(C≦30)}, aryl_{(C≦30)}, aralkyl_{C≦30 )}, acyl_{(C≦30)}, or a substituted version of any of these groups;
R₂ is hydroxy, peroxy or oxo;
R₃ is absent or methyl, provided that R₃ is absent when the atom to which is bound is part of double bond, further provided that when R₃ is absent the atom to which it is bound is part of a double bond;
R₄ is absent or methyl, provided that R₄ is absent when the atom to which is bound is part of double bond, further provided that when R₄ is absent the atom to which it is bound is part of a double bond; and
R₅ is:
   hydrogen, a monosaccharide group, a disaccharide group, an oligosaccharide group or a terpenoid group, or
   alkyl_{(C≦30)}, alkenyl_{(C≦30)}, alkynyl_{(C≦30)}, aryl_{(C≦30)}, aralkyl_{(C≦30)}, acyl_{(C≦30)}, or a substituted version of any of these groups;
      or a pharmaceutically acceptable salt, acetal, ketal or tautomer thereof.

In some embodiments, R₁ is a monosaccharide group, a disaccharide group, or an oligosaccharide group. In some embodiments, R₁ is a trisaccharide group, for example:

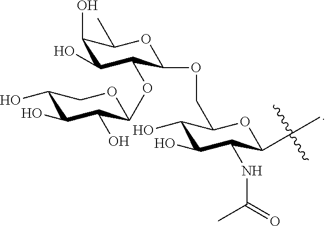

In some embodiments, R₂ is hydroxy. In other embodiments, R₂ is peroxy. In other embodiments, R₂ is oxo.

In some embodiments, R₃ is methyl. In other embodiments, R₃ is absent.

In some embodiments, R₄ is methyl. In other embodiments, R₄ is absent.

In some embodiments, R₅ is a monosaccharide group, a disaccharide group, or an oligosaccharide group, for example:

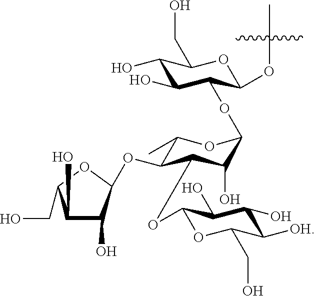

In some embodiments, the compound is selected from the group consisting of:

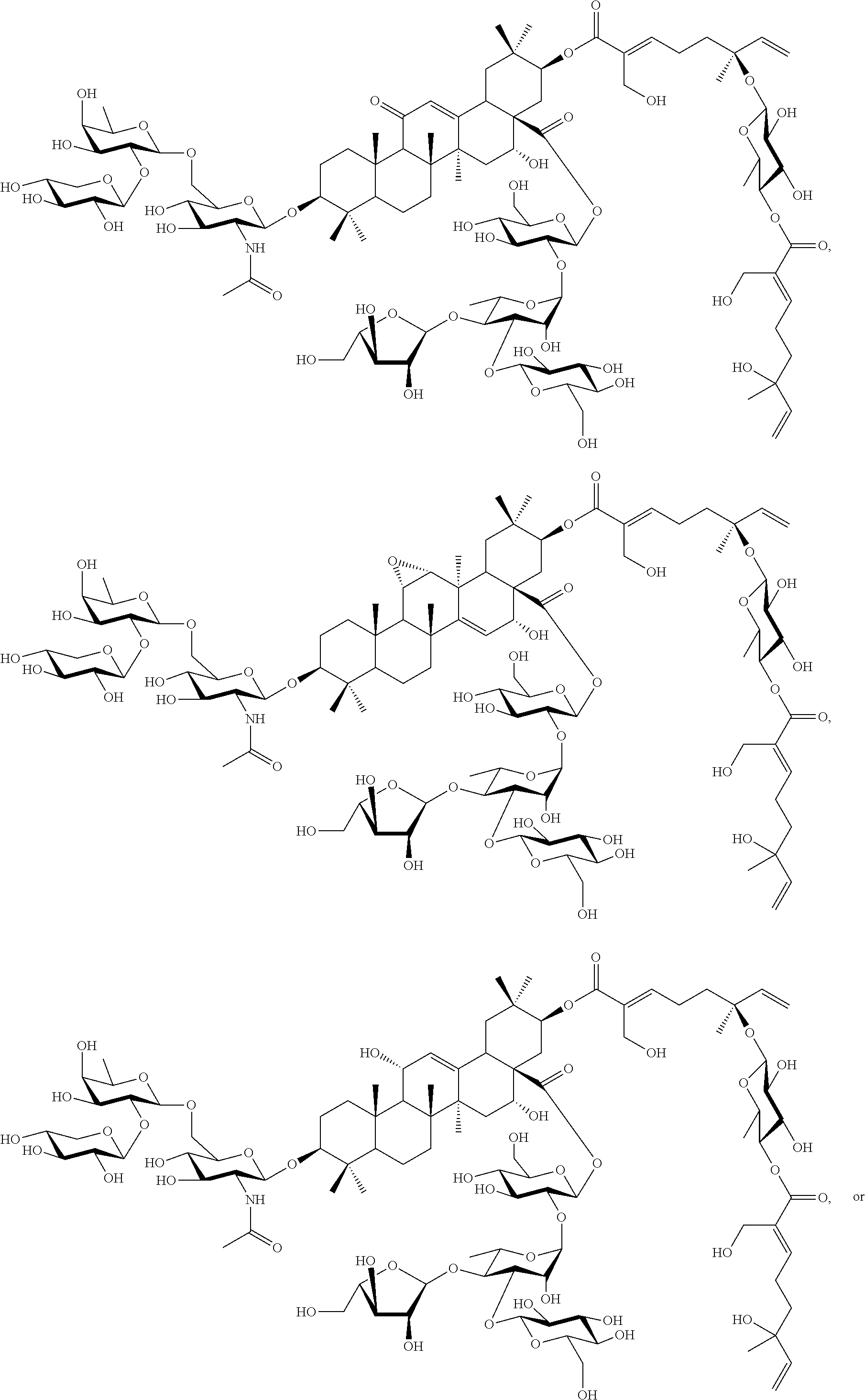

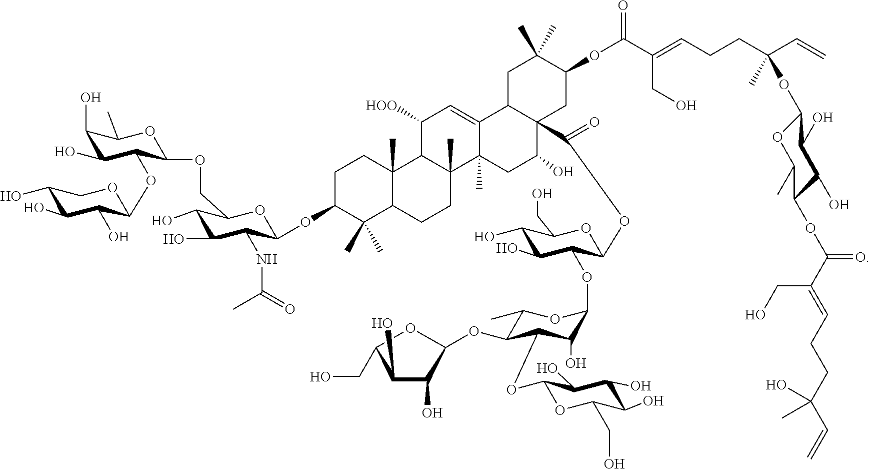

In another aspect, there are provided pharmaceutical compositions comprising a compound defined above and a pharmaceutically acceptable carrier. In some embodiments, the composition is formulated for oral administration. In some embodiments, the pharmaceutical composition further comprises one or more pharmaceutically acceptable excipients. In some embodiments, the composition is formulated for controlled release.

In another aspect, there are provided methods of treating proliferative disorders comprising administering to a patient in need thereof an effective amount of a compound defined above. In some embodiments, the proliferative disorder is cancer.

In another aspect, there are provided methods of treating inflammatory disorders comprising administering to a patient in need thereof an effective amount of a compound defined above.

In another aspect, there are provided methods of treating diseases or disorders associated with chemotaxis comprising administering to a patient in need thereof an effective amount of a compound defined above. In some embodiments, the disease or disorder is selected from the group consisting of autoimmune diseases and irritable bowel syndrome.

In another aspect, there are provided methods of treating metabolic disorders comprising administering to a patient in need thereof an effective amount of a compound defined above. In some embodiments, the metabolic disorder is obesity, diabetes, and combinations thereof.

In another aspect, there are provided kits comprising one or more compounds defined above.

In another aspect, there are provided methods of synthesizing any of the compounds defined above, comprising reacting Avicin D with one or more reagents to form a compound defined above.

The avicin derivatives described herein, and optional one or more additional active agents, can be combined with one or more pharmaceutically acceptable excipients and formulated for enteral, parenteral, topical, or pulmonary administration. Suitable oral dosage forms include, but are not limited to, tablets, caplets, capsules, syrups, solutions, suspensions, and emulsions. Suitable injectable formulations include solutions and suspensions. Suitable topical formulations include lotions, creams, ointments, and patches. Suitable pulmonary formulations include solution, suspensions, or aerosols which can be inhaled into the lung.

The compounds described herein may be used to treat a variety of diseases or disorders. Exemplary disorders include proliferative disorders, such as cancer; metabolic disorders, such as diabetes, diseases and disorders associated with chemotaxis, such as autoimmune diseases and irritable bowel syndrome, and combinations thereof.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

FIG. 1A is the steroidal backbone. FIG. 1B is the structure of avicin D.

FIGS. 2A & 2B are graphs showing the ability of avicins to inhibit NF-KB and activate NF-E2-related factor 2 (Nrf2). FIG. 2A shows that avicin D inhibits activation of NF-κB in a similar manner to dexamethasone (Dex). FIG. 2B shows that avicin D suppresses the expression of both constitutive and TNF-induced IL-6.

FIG. 3 is graph showing the expression of glucocorticoid receptor (GR) in HEK293T cells with and without GR.

FIG. 4 is graph showing that avicin D does not enhance phosphorylation of Ser-211 even after 16 hours.

FIG. 5 shows the formula of C-11 Hydroxy Avicin D [ALB 154491]. The dashed box highlights the hydroxy group.

FIG. 6 shows the formula of Oxidatively-Rearranged Avicin D [ALB 153752-2], with modifications in the C and D rings. These are highlighted by arrows 1-3.

FIG. 7 shows the formula of C-11 Keto Avicin D [ALB-153384]. The dashed box highlights the oxo group.

FIG. 8 shows the formula of C-11 Hydroperoxy Avicin D [ALB-154737]. The dashed box highlights the peroxy group.

FIG. 9 shows NF-κB activation results for some of the compounds of the present invention. Jurkat cells (2×10⁶/ml) were treated with avicin D or one of the derivatives defined in Table 1 (1 μM each) for 16 hrs. Cells were next exposed to TNF (1 nM) for 15 minutes. NF-κB activation was studied using p65 ab and the Trans AM NF-κB assay kit from Active motif. The results are presented as % of untreated, which in turn was taken as 100% activation.

FIG. 10 shows the activation of Nrf2 in response to treatment with some of the compounds from the present invention. Avicin D and t-BHQ have been used as positive controls. HepG2 (hepatocarinoma) cells were treated with 1 μM of each of the compounds. Nrf2 protein was detected in the cell lysates using western blot analysis. Intensities of protein bands were quantitated using NIH Image and these results have been shown in FIG. 10.

FIG. 11 shows the activation of MAP kinase in response to treatment with some of the compounds from the present invention. The ability of avicin D and the different analogues to activate MAPK in PC3 cells was analyzed by western blot analysis.

### DETAILED DESCRIPTION OF THE INVENTION

Disclosed herein are Avicin D derivatives, methods for their manufacture, and methods for their use, including for the treatment and/or prevention of cancer or other diseases.

### I. DEFINITIONS

When used in the context of a chemical group, “hydrogen” means —H; “hydroxy” means —OH; “peroxy” means —OOH; “oxo” means ═O; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylimino); “cyano” means —CN; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); and “sulfinyl” means —S(O)— (see below for definitions of groups containing the term sulfinyl, e.g., alkylsulfinyl).

The symbol “—” means a single bond, “═” means a double bond, and “≡” means triple bond. The symbol “- - - -” represents an optional bond, which if present is either single or double. The symbol “ [symbol] ” represents a single bond or a double bond. Thus, for example, the structure

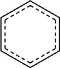

includes the structures

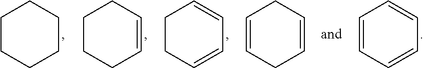

As will be understood by a person of skill in the art, no one such ring atom forms part of more than one double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation (e.g., either R or S) or the geometry is undefined (e.g., either E or Z).

Any undefined valency on an atom of a structure shown in this application implicitly represents a hydrogen atom bonded to the atom. When a group “R” is depicted as a “floating group” on a ring system, for example, in the formula:

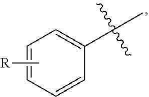

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

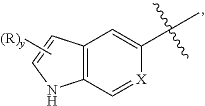

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “saturated” as used herein means the compound or group so modified has no carbon-carbon double and no carbon-carbon triple bonds, except as noted below. The term does not preclude carbon-heteroatom multiple bonds, for example a carbon oxygen double bond or a carbon nitrogen double bond. Moreover, it does not preclude a carbon-carbon double bond that may occur as part of keto-enol tautomerism or imine/enamine tautomerism.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl). When the term “aliphatic” is used without the “substituted” modifier only carbon and hydrogen atoms are present. When the term is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃.

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—,

### —CH₂C(CH₃)₂CH₂—, —CH₂CH₂CH₂—, and

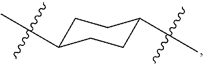 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. When any of these terms is used with the “substituted” modifier one or more hydrogen atom has been independently replaced by —OH, —F, —Cl, —Br, —I, —NH₂, ^{—}NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups. The term “hydroxyalkyl” is a subset of substituted alkyl, in which one or more hydrogen has been substituted with a hydroxy group and no other atoms aside from carbon, hydrogen and oxygen are present. The group —CH₂OH is a non-limiting example of a hydroxyalkyl group. An “alkane” refers to the compound H—R, wherein R is alkyl.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

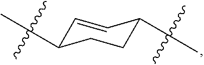 [SMILES: *C1C=CC(*)CC1]

are non-limiting examples of alkenediyl groups.

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃. The groups, —CH═CHF, —CH═CHCl and —CH═CHBr, are non-limiting examples of substituted alkenyl groups. An “alkene” refers to the compound H—R, wherein R is alkenyl.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. The term “alkynediyl” when used without the “substituted” modifier refers to a divalent unsaturated aliphatic group, with two carbon atoms as points of attachment, a linear or branched, cyclo, cyclic or acyclic structure, at least one carbon-carbon triple bond, and no atoms other than carbon and hydrogen. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃. An “alkyne” refers to the compound H—R, wherein R is alkynyl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl group (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and the monovalent group derived from biphenyl. The term “arenediyl” when used without the “substituted” modifier refers to a divalent aromatic group, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of arenediyl groups include:

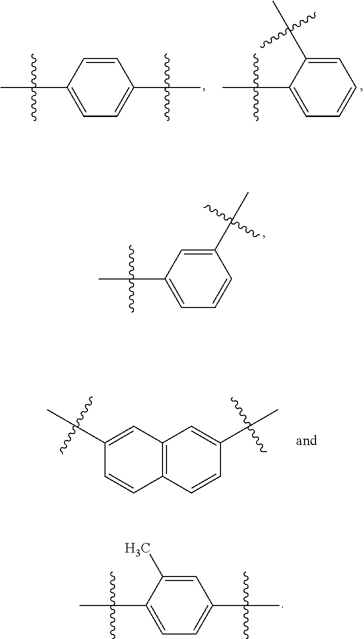

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃. An “arene” refers to the compound H—R, wherein R is aryl.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term is used with the “substituted” modifier one or more hydrogen atom from the alkanediyl and/or the aryl has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the group consists of no atoms other than carbon, hydrogen, aromatic nitrogen, aromatic oxygen and aromatic sulfur. As used herein, the term does not preclude the presence of one or more alkyl group (carbon number limitation permitting) attached to the aromatic ring or any additional aromatic ring present. Non-limiting examples of heteroaryl groups include furanyl, imidazolyl, indolyl, indazolyl (Im), methylpyridyl, oxazolyl, pyridyl, pyrrolyl, pyrimidyl, pyrazinyl, quinolyl, quinazolyl, quinoxalinyl, thienyl, and triazinyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of heteroarenediyl groups include:

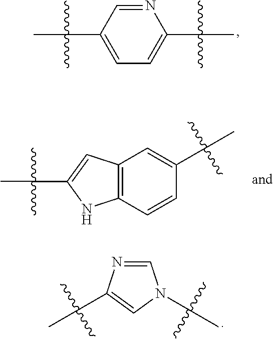 [SMILES: *c1ccc(*)nc1 ; *c1ccc2[nH]c(*)cc2c1 ; *c1cn(*)cn1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. When either of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃. The groups, —C(O)CH₂CF₃, —CO₂H (carboxyl), —CO₂CH₃ (methylcarboxyl), —CO₂CH₂CH₃, —C(0)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The terms “alkenyloxy”, “alkynyloxy”, “aryloxy”, “aralkoxy”, “heteroaryloxy”, and “acyloxy”, when used without the “substituted” modifier, refers to groups, defined as —OR, in which R is alkenyl, alkynyl, aryl, aralkyl, heteroaryl, and acyl, respectively. Similarly, the term “alkylthio” when used without the “substituted” modifier refers to the group —SR, in which R is an alkyl, as that term is defined above. The term “alkoxydiyl” when used without the “substituted” modifier refers to the divalent group —O-alkanediyl-. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃. The term “alcohol” corresponds to an alkane, as defined above, wherein at least one of the hydrogen atoms has been replaced with a hydroxy group.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, and “alkylsulfonylamino” when used without the “substituted” modifier, refers to groups, defined as —NHR, in which R is alkoxy, alkenyl, alkynyl, aryl, aralkyl, heteroaryl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, ^{—}N(CH₃)₂, —C(O)NH₂ or —OC(O)CH₃. The groups —NHC(O)OCH₃ and —NHC(O)NHCH₃ are non-limiting examples of substituted amido groups.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” when used without the “substituted” modifier refers to the group —OP(O)(OR)(OR'), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃.

The terms “alkylsulfonyl” and “alkylsulfinyl” when used without the “substituted” modifier refers to the groups —S(O)₂R and —S(O)R, respectively, in which R is an alkyl, as that term is defined above. The terms “alkenylsulfonyl”, “alkynylsulfonyl”, “arylsulfonyl”, “aralkylsulfonyl”, and “heteroarylsulfonyl”, are defined in an analogous manner. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃.

“Aglycone”, as used herein, generally refers to the pentacyclic core of an avicin, exclusive of any carbohydrate and terpenoid groups.

“C-3 glycone”, as used herein, refers to the sugar group at C-3 of the aglycone core.

“C-28 glycone”, as used herein, refers to the glycoside substituent at the C-28 carbonyl of the aglycone core.

“C-21 glycone”, as used herein, refers to the terpenoid-glycoside substituent at

C-21 of the aglycone core. The C-21 terpenoid glycoside of Avicin D contains two monoterpene groups, referred to herein as MT₁ or inner monoterpene group and MT₂ outer monoterpene group.

The term “glycoside” refers to a compound in which a sugar group is bound to a non-carbohydrate moiety. Typically the sugar group (glycone) is bonded through its anomeric carbon to another group (aglycone) via a glycosidic bond that has an oxygen, nitrogen or sulfur atom as a linker.

A “simple sugar” are the basic structural units of carbohydrates, which cannot be readily hydrolyzed into simpler units. The elementary formula of a simple monosaccharide is CₙH₂ₙOₙ, where the integer n is at least 3 and rarely greater than 7. simple monosachharides may be named generically according on the number of carbon atoms n: trioses, tetroses, pentoses, hexoses, etc. Simple sugars may be open chain (acyclic), cyclic or mixtures thereof. In these cyclic forms, the ring usually has 5 or 6 atoms. These forms are called furanoses and pyranoses, respectively—by analogy with furan and pyran. Simple sugars may be further classified into aldoses, those with a carbonyl group at the end of the chain in the acyclic form, and ketoses, those in which the carbonyl group is not at the end of the chain. Non-limiting examples of aldoses include: glycolaldehyde, glyceraldehydes, erythrose, threose, ribose, arabinose, xylose, lyxose, allose, altrose, glucose, mannose, gulose, idose, galactose and talose. Non-limiting examples of aldoses include: dihydroxyacetone, erythrulose, ribulose, xylulose, fructose, psicose, sorbose and tagatose. The ‘D-’ and ‘L-’ prefixes may be used to distinguish two particular stereoisomers which are mirror-images of each other. The term simple sugar also covers O-acetyl derivatives thereof.

A “amino sugar” refers to a derivative of a sugar, deoxy sugar, sugar acid or sugar alcohol, where one or more hydroxy group(s) has been replace with one more amino group(s). A “simple amino sugar” refers to a derivative of a simple sugar, simply deoxy sugar, simply sugar acid or sugar alcohol, where one or more hydroxy group(s) has been replace with one more amino group(s). These terms also cover N- and O-acetyl derivatives thereof. Non-limiting examples include N-acetylglucosamine, galactosamine, glucosamine and sialic acid.

The term “deoxy sugar” refers to a sugar derivative where one of the hydroxy groups of a carbohydrate has been replaced with a hydrogen atom. A “simple deoxy sugar” is a deoxy sugar derived from a simple sugar, as defined herein. These terms also cover O-acetyl derivatives thereof. Non-limiting examples of simple deoxy sugars are deoxyribose (based upon ribose), fucose, and rhamnose.

The term “sugar acid” refers to a sugar derivative where an aldehyde functional group or one or more hydroxy functional groups has been oxidized to a carboxyl group. Aldonic acids are those in which the aldehyde functional group of an aldose has been oxidized. Ulosonic acids are those in which the first hydroxyl group of a 2-ketose has been oxidized creating an α-ketoacid. Uronic acids are those in which the terminal hydroxyl group of an aldose or ketose has been oxidized. Aldaric acids are those in which both ends of an aldose have been oxidized. Non-limiting aldonic acids include glyceric acid (3C), xylonic acid (5C), gluconic acid (6C), and ascorbic acid (6C, unsaturated lactone). Non-limiting examples of ulosonic acids include neuraminic acid (5-amino-3,5-dideoxy-D-glycero-D-galacto-non-2-ulosonic acid) and ketodeoxy-octulosonic acid (KDO or 3-deoxy-D-manno-oct-2-ulosonic acid). Non-limiting examples of uronic acids include glucuronic acid (6C), galacturonic acid (6C), and iduronic acid (6C). Non-limiting example of aldaric acids include tartaric acid (4C), meso-galactaric acid (mucic acid) (6C), and D-glucaric acid (saccharic acid) (6C). A “simple sugar acid” is a sugar acid derived from a simple sugar. These terms also cover O-acetyl derivatives thereof.

The term “sugar alcohol” refers to a sugar derivative whose carbonyl group (aldehyde or ketone, reducing sugar) has been reduced to a primary or secondary hydroxyl group. Non-limiting examples of sugar alcohols include: glycol (2-carbon), glycerol (3-carbon), erythritol (4-carbon), threitol (4-carbon), arabitol (5-carbon), xylitol (5-carbon), ribitol (5-carbon), mannitol (6-carbon), sorbitol (6-carbon), dulcitol (6-carbon), iditol (6-carbon), isomalt (12-carbon), maltitol (12-carbon), lactitol (12-carbon) or polyglycitol. A “simple sugar alcohol” is a sugar alcohol derived from a simple sugar. These terms also cover O-acetyl derivatives thereof.

As used herein, the term “monosaccharide group” refers to a monovalent carbohydrate group, with a carbon atom as the point of attachment. The term covers the groups resulting from removal of a hydroxyl radical from a simple sugar (e.g., glucose), simple deoxy sugar (e.g., fucose), simple sugar acid (e.g., gluconic acid), simple sugar alcohol (e.g., xylitol) or simple amino sugar (e.g., glucosamine). Typically the monosaccharide group is bonded through its anomeric carbon to another group (aglycone) via oxygen atom linker. In some cases the linker may be a nitrogen or sulfur atom.

A “disaccharide group” is a monovalent carbohydrate group consisting of two monosaccharide groups, wherein the second monosaccharide group replaces a hydrogen on a hydroxy group of the first monosaccharide group. Non-limiting examples of disaccharide groups include those derived from sucrose, lactulose, lactose, maltose trehalose and cellobiose.

A “trisaccharide group” is a monovalent carbohydrate group consisting of three monosaccharide groups, wherein the second monosaccharide group replaces a hydrogen on a hydroxy group of the first monosaccharide group and the third monosaccharide group replaces a hydrogen on a hydroxy group of either the first or the second monosaccharide groups.

An oligosaccharide is a monovalent carbohydrate group consisting of three to ten, preferably three to six monosaccharide groups, wherein the second monosaccharide replaces a hydrogen on a hydroxy group of the first monosaccharide, the third monosaccharide replaces a hydrogen on a hydroxy group of either the first or the second monosaccharide groups, and subsequent monosaccharide groups replace hydrogens on any previously joined monosaccharide groups, thus forming either a linear or branched structure.

A terpenoid group is a monovalent radical derived from removing a hydrogen from a terpene, that is from compound derived from the biosynthesis of isoprene and having the molecular formula (C₅H₈)ₙ, wherein n is 1, 2, 3, 4, 5, 6, 7 or 8.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo [2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methane-sulfonates, ethanesulfonates, benzenesulfonates, p-toluenesulfonates, cyclohexyl-sulfamates, quinates, esters of amino acids, and the like. Similarly, a compound comprising an amine group may be administered as an amide that is converted by hydrolysis in vivo to the amine compound.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≦15%, more preferably ≦10%, even more preferably ≦5%, or most preferably ≦1% of another stereoisomer(s).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like.

Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Effective amount,” “Therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, the term “water soluble” means that the compound dissolves in water at least to the extent of 0.010 mole/liter or is classified as soluble according to literature precedence.

Other abbreviations used herein are as follows: DMSO, dimethyl sulfoxide; NO, nitric oxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFNγ or IFN-γ, interferon-γ; LPS, bacterial endotoxic lipopolysaccharide; TNFα or TNF-α, tumor necrosis factor-α; IL-1β, interleukin-1β; GAPDH, glyceraldehyde-3-phosphate dehydrogenase; MTT, 3- [4,5-dimethylthiazol-2-yl]-2,5-diphenyltetrazolium bromide; TCA, trichloroacetic acid; HO-1, inducible heme oxygenase.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. CYTOPROTECTIVE dERIVATIES OF AVICIN-D

The compounds provided by the present disclosure may be made using the methods outlined below and further described in the Examples section. Precursor molecules used to make compounds described herein can be isolated from extracts of the species Acacia victoriae. Methods of extracting triterpene compositions and avicins are described in U.S. Pat. No. 6,444,233 to Arntzen et al., which are incorporated herein by reference. The avicin derivatives described herein may be prepared by the biocatalysis of such avicin-containing extracts. Generally, one or more starting materials, such as Avicin D, is reacted with one or more enzymes and the resulting products are analyzed. Suitable enzymes include hydrolases (e.g., glycosidases, proteases, esterases, acylases, and lipases); laccases and oxidase/mediator combinations; peroxidases, haloperoxdases, and lipoxygenases.

Alternatively, the avicin derivatives described can be prepared synthetically or semi-synthetically from a naturally occurring precursor component, such as Avicin D, or another suitable starting material. Such methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereorneric mixtures and individual diastereomers. In some embodiments, a single diastereomer is obtained. The compounds can be formulated as a mixture of one or more diastereomers. Alternatively, the diastereomers can be separated and one or more of the diastereomers can be formulated individually. The chiral centers of the compounds of the present invention can have the S or the R configuration, as defined by the IUTPAC 1974 Recommendations. For example, mixtures of stereoisomers may be separated using the techniques taught in the Examples section below, as well as modifications thereof.

Atoms making up the compounds of the present invention are intended to include all isotopic forms of such atoms. Compounds of the present invention include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Compounds of the present invention may also exist in prodrug form. Since prodrugs are known to enhance numerous desirable qualities of pharmaceuticals (e. g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound, Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present invention include those that have been further modified to comprise substituents that are convertible to hydrogen in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such aβ, β, β,-trichloroethoxycarbonyl and β-iodoethoxycarbonyl).

The compounds described herein may exist in unsolvated forms as well as solvated forms, including hydrated forms. In general, the solvated forms are equivalent to unsolvated forms and are within the scope of the compounds described herein. The compounds described herein may exist in multiple crystalline or amorphous forms. In general, all physical forms are equivalent for the uses described herein and are intended to be within the scope of the compounds described herein.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

### III. METHODS OF USING AVICIN-D DERIVATIVES

The compounds described herein can be administered to provide an effective amount to treat a variety of diseases and disorders, such as proliferative disorders (e.g., cancers), metabolic disorders (e.g., obesity and diabetes), diseases associated with chemotaxis (e.g., autoimmune disorders, infections, irritable bowel syndrome), and combinations thereof. The therapeutically effective doses are readily determinable using an animal model, as described in U.S. Pat. No. 6,444,233. For example, experimental animals bearing solid tumors are frequently used to optimize appropriate therapeutic doses prior to translating to a clinical environment. Such models are known to be reliable in predicting effective anti-cancer strategies.

In certain embodiments, it may be desirable to provide continuous delivery of one or more avicin derivatives to a patient in need thereof. For intravenous or intraarterial routes, this can be accomplished using drip systems, such as by intravenous administration. For topical applications, repeated application can be done or a patch can be used to provide continuous administration of the avicin derivatives over an extended period of time. Extended release formulations can also be used that provide limited but constant amounts of the drug over an extended period of time.

For internal applications, continuous perfusion of the region of interest may be desirable. This could be accomplished by catheterization, post-operatively in some cases, followed by continuous administration of the one or more avicin derivatives. The time period for perfusion can be readily determined by the attending physician clinician for a particular patient. Perfusion times typically range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered.

The compositions described herein contain an effective amount of the one or more avicin derivatives. The amount to be administered can be readily determined by the attending physician based on a variety of factors including, but not limited to, age of the patient, weight of the patient, disease or disorder to be treated, presence of a pre-existing condition, and dosage form to be administered (e.g., immediate release versus modified release dosage form). Typically, the effective amount is from about 0.1 mg/kg/day to about 100 mg/kg/day, more preferably from 0.1 mg/kg/day to 50 mg/kg/day, more preferably from 0.1 mg/kg/day to 25 mg/kg/day, and most preferably from 0.1 mg/kg/day to 10 mg/kg/day. Dosages greater or less than this may be administered depending on the diseases or disorder to be treated. For example, preliminary data suggests that avicins, such as Avicin D, are effective at inhibiting chemotaxis at picomolar and nanomolar concentrations, as discussed below.

A. Proliferative Disorders (e.g., Cancer)

In the concept of proliferative diseases, the avicin derivatives described herein can be administered to a subject in need thereof to treat the subject either prophylactically (e.g., to prevent cancer) or therapeutically (e.g., to treat cancer after it has been detected), including reducing tumor growth, reducing the risk of local invasiveness of a tumor, increasing survival time of the patient, and/or reducing the risk of metastasis of a primary tumor. In some embodiments, the compounds described herein may be used to contact a target cell to inhibit the initiation and promotion of cancer, to kill cancer/malignant cells, to inhibit cell growth, to induce apoptosis, to inhibit metastasis, to decrease tumor size, to otherwise reverse or reduce the malignant phenotype of tumor cells, and combinations thereof. In some embodiments, this is achieved by contacting a tumor or tumor cell with a single composition or pharmacological formulation that includes the avicin derivative(s), or by contacting a tumor or tumor cell with more than one distinct composition or formulation, simultaneously, wherein one composition includes one or more avicin derivatives described herein and the other includes a second agent.

Examples of cancers which can be treated include, but are not limited to, cancer of the skin, colon, uterine, ovarian, pancreatic, lung, bladder, breast, renal system, and prostate. Other cancers include, but are not limited to, cancers of the brain, liver, stomach, esophagus, head and neck, testicles, cervix, lymphatic system, larynx, esophagus, parotid, biliary tract, rectum, endometrium, kidney, and thyroid; including squamous cell carcinomas, adenocarcinomas, small cell carcinomas, gliomas, neuroblastomas, and the like. Assay methods for ascertaining the relative efficacy of the compounds described herein in treating the above types of cancers as well as other cancers are well known in the art.

In some embodiments, the compounds described herein may also be used to treat metastatic cancer either in patients who have received prior chemo, radio, or biological therapy or in previously untreated patients. In one embodiment, the patient has received previous chemotherapy. For example, patients can be treated using a variety of routes of administration including systemic administration, such as intravenous administration or subcutaneous administration, oral administration or by intratumoral injection. The pharmaceutical dose(s) administered would preferably contain between 10 and 25 mg of avicins per kg of patient body weight per day, including about 13, 16, 19, and 22 mg/kg/day. Alternatively, the patient could be treated with one or more pharmaceutical compositions comprising from about 1 mg/kg/day of the avicins of the invention to about 100 mg/kg/day, including about 3, 6, 9, 12, 15, 18, 21, 28, 30, 40, 50, 60, 70, 80 and 90 mg/kg/day of the avicins described herein.

The treatment course typically consists of daily treatment for a minimum of eight weeks or one injection weekly for a minimum of eight weeks. Upon election by the clinician, the regimen may be continued on the same schedule until the tumor progresses or the lack of response is observed.

The avicin derivatives described herein can also be used to treat patients who have been rendered free of clinical disease by surgery, chemotherapy, and/or radiotherapy. In these aspects, the purpose of therapy is to prevent or reduce the likelihood of recurrent disease. Adjuvant therapy can be administered in the same regimen as described above to prevent recurrent disease.

The avicin derivatives described herein can also be used to target and/or kill cancer stem cells (CSCs). Recent studies have shown the existence of self renewing, stem-like cells within tumors, now called cancer stem cells (CSCs). Reya et al., 2001, which is incorporated herein by reference. CSCs are resistant to most anti cancer treatments and possess the ability to seed new tumors. Based on the cytotoxicity results using a CSC model (Example 4), the avicin deriviates may be used to kill cancer stem cells (CSCs).

B. Anti-Inflammatory Disorders

The compounds described herein can also be used as anti-inflammatory agents. Avicin D has been shown to be an inhibitor of transcription factor NF-KB, which plays an important role in the inflammatory response. This finding is particularly significant given the increasing amount of evidence suggesting the central role of inflammatory response in carcinogenesis. Treatment of patients with the avicins described herein may, therefore, potentially alleviate a wide degree of ailments associated with inflammation, including tumorigenesis and tissue damage.

The initial stages of an inflammatory response are characterized by increased blood vessel permeability and release (exudation) of histamine, serotonin and basic polypeptides and proteins. This is accompanied by hyperaemia and oedema formation. Subsequently, there is cellular infiltration and formation of new conjunctive tissue. It is believed that treatment with the compounds of the invention can limit these early stages of inflammation and, thereby, decrease the negative effects associated with the inflammatory condition.

In one embodiment, one or more avicin derivatives are administered as non-steroidal selective glucocorticoid receptor modulators, alone or in combination with one or more glucocorticoids. Glucocorticoids (GCs) are essential steroid hormones, secreted by the adrenal cortex, that play a critical role in the maintenance of homeostasis in mammals. GCs are involved in the regulation of development, metabolism and stress responses. They are also known to be potent immunosuppressive, anti-allergic, and anti-inflammatory drugs.

GCs exert their effects via the glucocorticoid receptor (GR), a cytoplasmic transcription factor belonging to the superfamily of thyroid/steroid nuclear hormone receptors. Upon binding of a ligand, the GR is released from an inactive cytoplasmic complex, and translocates into the nucleus. In the nucleus, GR binds as a homodimer to consensus sequences, termed GC response elements (GREs), in the promoter region of GC-sensitive genes to induce transcription (transactivation), of various genes such as those encoding tyrosine amino transferase (TAT), some key enzymes of glycolysis, lipid metabolism and immune response. Another important mechanism of GR-mediated transcriptional regulation involves repression (transrepression) of transcription, and is mediated through GR-protein interactions.

For its transrepressive action, GR binds as a monomer to transcription factors, such as NF-KB, AP-1, Stat5 and others. Studies using transgenic mice harboring mutated GR have shown that the transrepressive activity of GRs is responsible for the anti-inflammatory actions of GCs. The desired anti-inflammatory and immunosuppressant effects of GCs, however, are most often accompanied by undesirable side effects, such as diabetes, obesity, hypertension, skin atrophy, and many others, most of which are believed to be mediated via transactivation. Studies showing that these two activities of GR are seperable has resulted in extensive efforts to identify ligands that preferentially induce the transrepression and not the transactivation function of GRs. Such ligands termed as “dissociated ligands” are likely to have immense therapeutic value with reduced side effects.

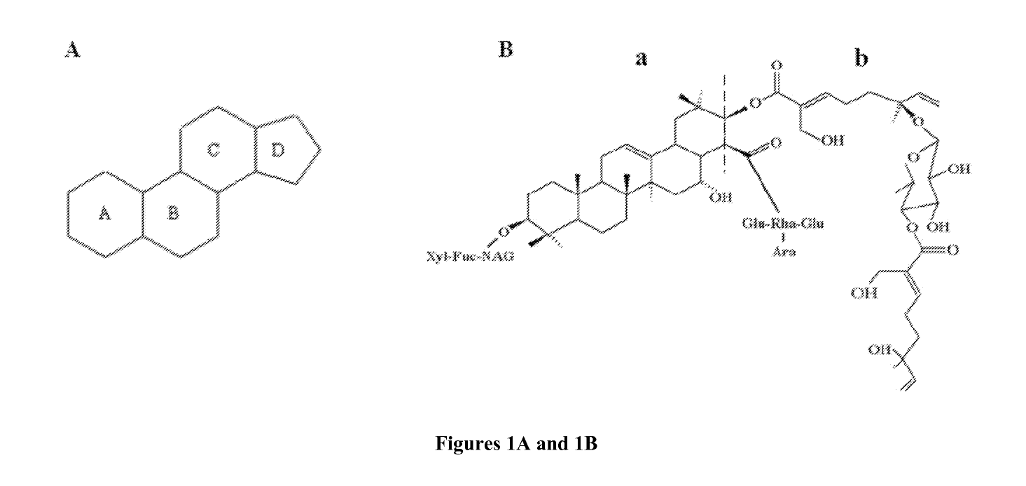

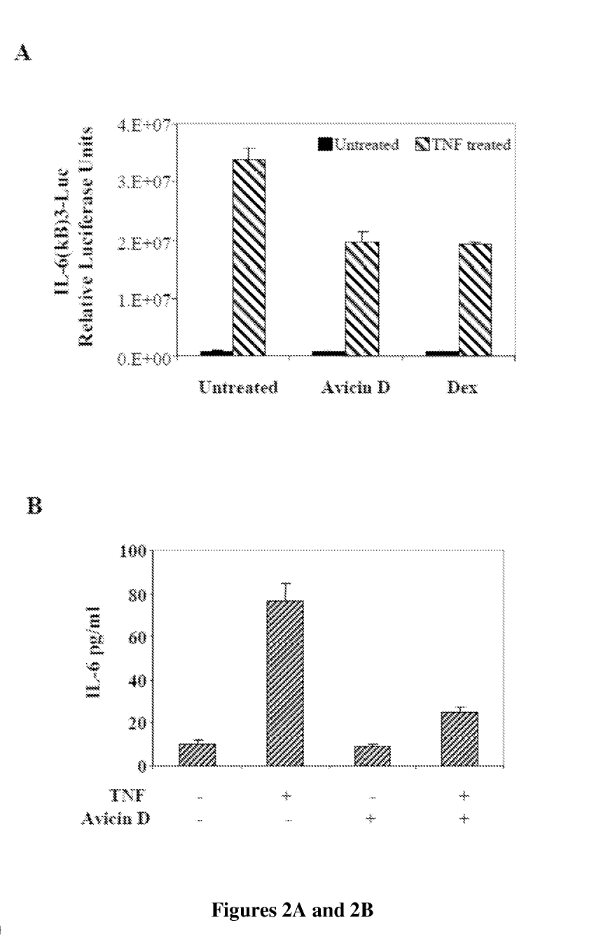

The pentacyclic backbone in the avicin derivatives described herein makes them structurally comparable to steroids (see FIGS. 1A & 1B). FIG. 1A is the steroidal backbone while FIG. 1B is Avicin D. Studies have shown that avicins are inhibitors of NF-KB and activate NF-E2-related factor 2 (Nrf2), accounting for their anti-inflammatory and stress responsive properties (see FIG. 2). FIG. 2A shows that Avicin D inhibits activation of NF-κB in a similar manner to dexamethasone. FIG. 2B shows that avicin D suppresses the expression of both constituitive and TNF-induced IL-6, in confirmation with the anti-inflammatory effects of avicins.

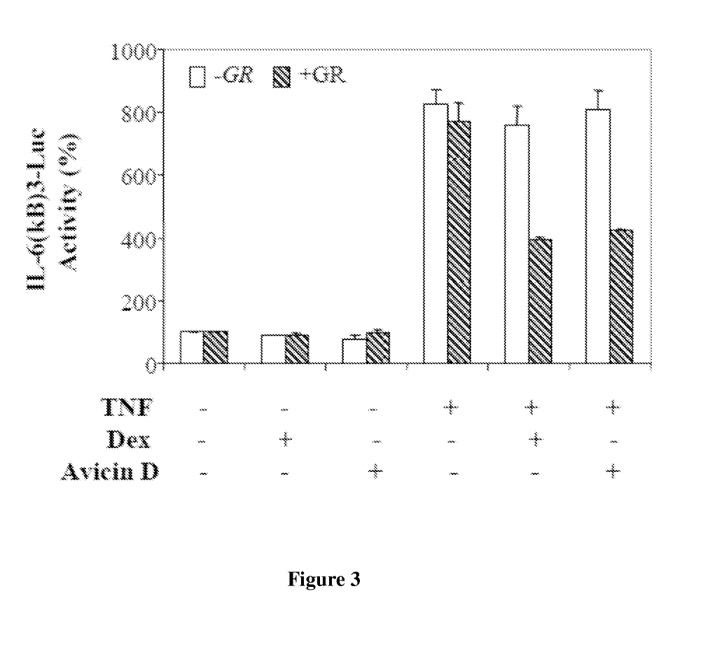

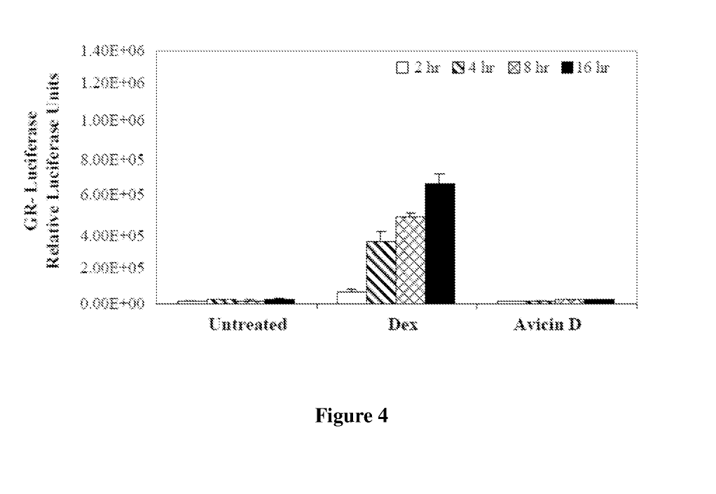

Previous studies have demonstrated that avicins can bind to GR and induce its nuclear translocation. This event is followed by inhibition of NF-KB activity via GR (see FIG. 3), while GR-driven transactivation itself is not induced, suggesting that avicins could act as dissociated ligands for GR (see FIG. 4). FIG. 3 shows the expression of GR in HEK293T cells with A549 and other cell lines. As shown in FIG. 3, neither avicin D nor dexamethasone had an effect on the luciferase activity in wild type HEK 293T cells. However, TNF-induced activation of p(IL6-κB)350hu.IL6P-Luc was inhibited by both avicin D and Dex in HEK 293T cells transfected with GR (FIG. 3). These results indicate that the presence of GR is required for avicin (as well as Dex) to downregulate the activation of NF-κB. FIG. 4 shows that avicin D does not enhance phosphorylation of Ser-211 even after 16 hours. In contrast, dexamethasone under similar conditions induced Ser-211 phosphorylation. This observation supports the notion that avicins are not able to induce GR transactivation.

Modeling of avicin-GR interaction revealed that the avicin molecule binds to the antagonist confirmation of the GR, which supports the finding that avicins can act as a dissociated GR ligand. Avicins can therefore be classified as nonsteroidal selective GR modulators. The use of avicins in place of, or in combination with one or more glucocorticoids, reduces the dosage of glucocorticoid administered and thus should minimize the adverse side effects associated with these compounds.

C. Metabolic Disorders

The avicins described herein may be used to treat metabolic disorders. Recent studies have shown that avicins regulate cellular energy metabolism and activated AMP-activated protein kinase (AMPK), a key regulator of fatty acid and glucose homeostasis. This suggests that the avicins described herein may be used to treat metabolic diseases including obesity, a burgeoning disorder that contributes to cardiovascular disease, and diabetes (including both type I and type II diabetes). The avicins described herein can also be used to treat insulin resistance and metabolic syndrome.

Avicin D has been shown to inhibit adipogenesis and reduce intracellular triglyceride level in a dose-dependent manner in 3T3-L1 cells. Avicin D suppressed preadipocyte differentiation, but did not affect adipolysis and adipocyte apoptosis. Avicin D inhibited PKA-mediated CREB activity, which in turn suppressed the expression of adipogenesis genes, such as CEBPs and PPAR-γ. Avicin D increased the expression and secretion of adiponectin in adipocytes. Importantly, avicin D inhibited the differentiation of human preadipocytes to mature adipocytes and reduced intracellular triglyceride levels as well. Taken together, these results suggest that avicins could serve as important metabolic regulators in the control of adipogenesis and treatment and prevention of metabolic syndromes.

As discussed above, avicins can inactivate NF-κB pathway and decrease IL-6, TNFα-mediated inflammatory reaction. In addition, avicins can also activate the proteins downstream of Nrf2, such as glutathione peroxidase, heme oxygenase, and thioredoxin reductase in vitro and in vivo, indicative of their antioxidant effects. These data suggest that avicins may be effective in adipogenesis control.

As shown in the examples below, avicins exhibit potent activity to inhibit adipogenesis in vitro, and reduced serum total cholesterol, triglyceride, and LDL levels, increased serum HDL concentration (p<0.05) in vivo. More importantly, applying these compounds to either undifferentiated fibroblasts or mature adipocytes does not produce any cytotoxic effects. Administering avicins to hamsters orally did not cause observable side effects in these animals. Thus it has been demonstrated that avicins are highly effective in inhibiting adipogenesis while having no short-term side effects, which makes avicins potentially useful candidates for obesity control.

D. Diseases and Disorders Associated with Chemotaxis

Chemotaxis is the phenomenon in which bodily cells, bacteria, and/or other single-cell or multicellular organisms direct their movements according to certain chemicals in their environment. In multicellular organisms, chemotaxis is critical to early (e.g. movement of sperm towards the egg during fertilization) and subsequent phases of development (e.g. migration of neurons or lymphocytes) as well as in normal function. In addition, it has been recognized that mechanisms that allow chemotaxis in animals can be subverted during cancer metastasis.

In one embodiment, one or more avicin derivatives are administered alone or in combination with an additional active agent, such as corticosteroid to inhibit chemotaxis, for example, the migration of immune cells or cancer cells. The compounds described herein may inhibit chemotaxis at picomolar to low nanomolar concentrations.

E. Other Uses

The compounds described herein can be also be used as anti-fungal and anti-viral agents, piscicides or molluscicides, contraceptives, antihelmintics, UV-protectants, expectorants, diuretics, anti-inflammatory agents, regulators of cholesterol metabolism, cardiovascular effectors, anti-ulcer agents, analgesics, sedatives, immunomodulators, antipyretics, angiogenesis regulators, agents for decreasing capillary fragility, agents to combat the effects of aging, and agents for improving cognition and memory.

The compounds described herein may be used to regulate angiogenesis, alone or in combination with one or more additional angiogenesis modulators. Angiogenesis or neovascularization is defined as the growth of new blood vessels. Tumors and cancers induce angiogenesis to provide a life-line for oxygen and nutrients for the tumor to thrive. The development of new blood vessels also provides exits for malignant cancer cells to spread to other parts of the body. Angiogenesis inhibition therefore benefits cancer patients. On the other hand, angiogenesis is required at times such as wound healing. These wounds can be external wounds or internal organ wounds that result from accidents, burns, injury and surgery. Thus, agents that promote angiogenesis have a great potential for use in therapy for wound healing.

The compounds described herein can be used to modulate cholesterol metabolism. In particular, the compounds described here may be used to lower the serum cholesterol levels of human patients. For the treatment of cardiovascular conditions, the compounds described herein can be used to treat arrhythmic action and further may be used as a vascular relaxant, resulting in antihypertensive activity.

The plant species from which the compounds of the invention were identified, Acacia victoriae, was selected, in part, because it is native to arid regions. An important function of the metabolism of plants from these regions is the production of compounds which protect cells from ultraviolet radiation. The compounds described herein can be used as UV-protectants. For example, suitable applications include the use of the avicins described herein as an ingredient in sunblock, or other similar lotions for application to human skin.

Other possible applications of the avicins described herein include protection in the central nervous system damage, in effect, memory loss or enhanced cognitive function, use as an antioxidant (monitoring blood levels of oxidative molecules), or increase of nitric oxide (NO), for the treatment of hypertension or atherosclerosis.

### IV. FORMULATIONS

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

A. Parenteral Formulations

The compounds described herein can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In one embodiment, the avicin derivative(s) are formulated in a carrier containing 5% dextrose, alone or in combination with 10% propylene glycol. In another embodiment, the avicins are formulated in 150 mMol NaCl solution and 10 mMol sodium acetate (pH adjusted to 4.5), optionally containing polysorbate 80. Formulations may be stable over a period of 6 months when stored at room temperature or 5° C., with the avicin purity averaging about 95%.

B. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition which may include plasticizers, pigments, colorants, stabilizing agents, and glidants. Delayed release dosage formulations may be prepared as described in standard references such as “Pharmaceutical dosage form tablets” (1989), “Remington—The science and practice of pharmacy” (2000), and “Pharmaceutical dosage forms and drug delivery systems” (1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA). Oral dosage forms, such as capsules, tablets, solutions, and suspensions, can for formulated for controlled release. For example, the one or more avicin derivatives and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more avicins and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more avicin derivatives, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the avicins and/or additional active agents.

C. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The compounds can also be formulated for intranasal delivery, pulmonary delivery, or inhalation. The compositions may further contain one or more chemical penetration enhancers, membrane permeability agents, membrane transport agents, emollients, surfactants, stabilizers, and combination thereof.

D. Pulmonary Formulations

In one embodiment, the avicin derivatives are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorbtion occurs. The alveoli are covered by a thin epithelium without cilia or a mucus blanket and secrete surfactant phospholipids (Patton and Platz, 1992).

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung (Gonda, 1990). The deep lung, or alveoli, are the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

### V. KITS

In various aspects, a kit is envisioned containing one or more compounds described herein. The kit may contain one or more sealed containers, such as a vial, containing any of the compounds described herein and/or reagents for preparing any of the compounds described herein. In some embodiments, the kit may also contain a suitable container means, which is a container that will not react with components of the kit, such as an Eppendorf tube, an assay plate, a syringe, a bottle, or a tube. The container may be made from sterilizable materials such as plastic or glass.

The kit may further include instructions that outline the procedural steps for methods of treatment or prevention of disease, and will follow substantially the same procedures as described herein or are known to those of ordinary skill The instruction information may be in a computer readable media containing machine-readable instructions that, when executed using a computer, cause the display of a real or virtual procedure of delivering a pharmaceutically effective amount of one or more compounds described herein.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the claims.

### Example 1

### Preparation of a C-11 Hydroxy Derivative of Avicin D and an Oxidatively-Rearranged Derivative of Avicin D

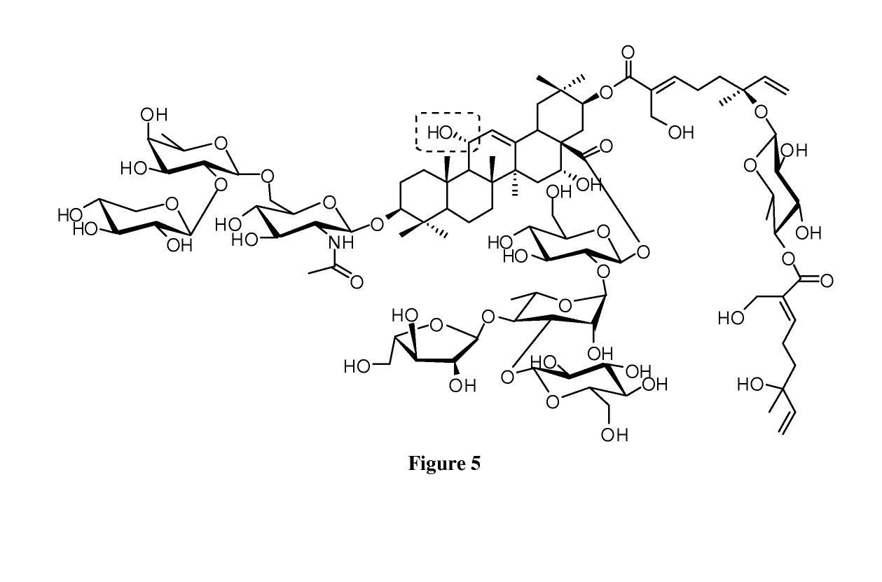

C-11 Hydroxy Avicin D [ALB 154491] corresponds to the formula shown in FIG. 5 and the following chemical name:
(3S,4aR,5R,6aS,6bR,10S,12aS,13R)-((2S,3S,4S,5S)-3-((2S,3R,4S,5S)-5-((2S,3S,5S)-3,4-dihydroxy-5-(hydroxymethyl)tetrahydrofuran-2-yloxy)-3-hydroxy-6-methyl-4-((2S,3S,4S,5S)-3,4,5-trihydroxy-6-(hydroxymethyl)tetrahydro-2H-pyran-2-yloxy)tetrahydro-2H-pyran-2-yloxy)-4,5-dihydroxy-6-(hydroxymethyl)tetrahydro-2H-pyran-2-yl) 10-((2R,3S,4R,5S)-3-acetamido-6-4(2R,3S,4S,5R)-4,5-dihydroxy-6-methyl-3-((2S,3S,4S,5R)-3,4,5-trihydroxytetrahydro-2H-pyran-2-yloxy)tetrahydro-2H-pyran-2-yloxy)methyl)-4,5-dihydroxytetrahydro-2H-pyran-2-yloxy)-3-((6S,E)-6-((2S,3S,4R,5S)-3,4-dihydroxy-5-(E)-6-hydroxy-2-(hydroxymethyl)-6-methylocta-2,7-dienoyloxy)-6-methyltetrahydro-2H-pyran-2-yloxy)-2-(hydroxymethyl)-6-methylocta-2,7-dienoyloxy)-5,13-dihydroxy-2,2,6a,6b,9,9,12a-heptamethyl-1,2,3,4,4a,5,6,6a,6b,7,8,8a,9,10,11,12, 12a,12b,13,14b-icosahydropicene-4a-carboxylate)

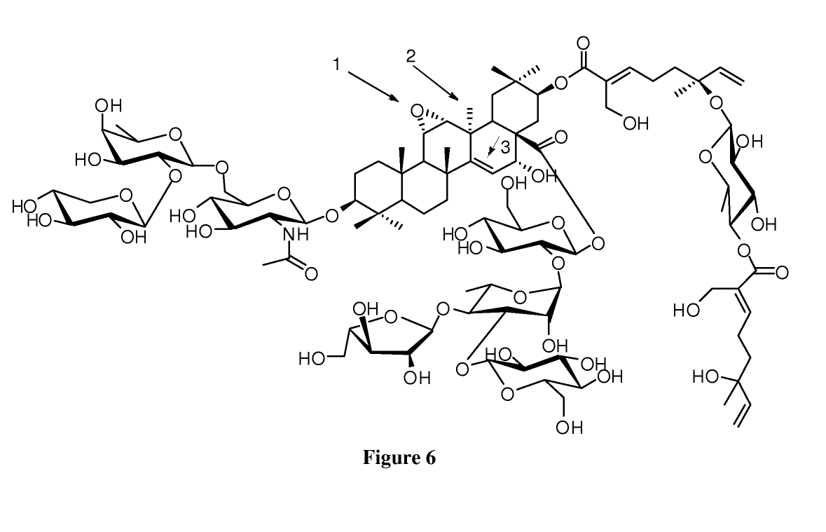

Oxidatively-Rearranged Avicin D [ALB 153752-2] corresponds to the formula shown in FIG. 6 and the following chemical name:
(3S,4aR,5R,6bR,10S,12aS,12cS,13aR,13bS)-((2S,3S,4S,5S)-3-((2S,3R,4S,5S)-5-((2S,3S,5S)-3,4-dihydroxy-5-(hydroxymethyl)tetrahydrofuran-2-yloxy)-3-hydroxy-6-methyl-4-((2S,3S,4S,5S)-3,4,5-trihydroxy-6-(hydroxymethyl)tetrahydro-2H-pyran-2-yloxy)tetrahydro-2H-pyran-2-yloxy)-4,5-dihydroxy-6-(hydroxymethyl)tetrahydro-2H-pyran-2-yl) 10-((2R,3S,4R,5S)-3-acetamido-6-(((2R,3S,4S,5R)-4,5-dihydroxy-6-methyl-3-((2S,3S,4S,5R)-3,4,5-trihydroxytetrahydro-2H-pyran-2-yloxy)tetrahydro-2H-pyran-2-yloxy)methyl)-4,5-dihydroxytetrahydro-2H-pyran-2-yloxy)-3-((6S ,E)-6-((2S ,3S ,4R,5S)-3,4-dihydroxy-5-((E)-6-hydroxy-2-(hydroxymethyl)-6-methylocta-2,7-dienoyloxy)-6-methyltetrahydro-2H-pyran-2-yloxy)-2-(hydroxymethyl)-6-methylocta-2,7-dienoyloxy)-5-hydroxy-2,2,6b,9,9,12a,13b-heptamethyl-1,2,3,4,4a,5,6b,7,8,8a,9,10,11,12,12a,12b,12c,13a,13b,13c-icosahydropiceno[13,14-b] oxirene-4a-carboxylate)
   These two compounds were synthesized as follows:

To a solution of Avicin D (0.25 g, 0.12 mmol) in methanol (5 mL) and 50 mM sodium citrate buffer (pH 5.5, 75 mL) was added 10 mL of a 50 mM violuric acid monohydrate solution in dimethyl sulfoxide and laccase enzyme (Europa bioproducts, 450 mg). The reaction mixture was incubated at 27 ° C., 100 rpm agitation for 18 h. The reaction mixture was diluted with water (200 mL) and loaded onto a pre-conditioned Alltech C18 SPE cartridge (10 g). The cartridge was washed with water (200 mL) and the products were eluted with methanol (200 mL). The methanol elute was concentrated to ca. 20 mL volume by evaporation under reduced pressure and the products were purified by preparative HPLC.

Column—Waters SunFire C18 OBD (150×50 mm, 5 μm)

Column temperature—Ambient

Solvents—A (water+0.1% formic acid); B (acetonitrile+0.1% formic acid)

Gradient—Linear (25% B to 30% B within 20 min then to 45% B within 10 min).

Flowrate—110 mL/min and λ220 nm

Pure fractions were combined and lyophilized to afford C-11 Hydroxy Avicin D (44 mg, 17%) and Oxidatively Rearranged Avicin D (51 mg, 20%) as white solids.

TABLE 1
Overview of Analysis of C-11 Hydroxy Avicin D

| TEST | RESULT/REFERENCE |
|---|---|
| Appearance | White solid |
| 500 MHz ¹H NMR | Assignments and spectra - Agree with |
| Spectrum (CD₃OD) | structure |
| 500 MHz ¹³C | Assignments and spectra - Agree with |
| NMR Spectrum | structure |
| 500 MHz ¹H NMR | Consistent |
| Spectrum (CD₃OD) | |
| LC-MS (TIS) | m/z 2121 [M + Na]⁺ |
| Analysis | |
| High Resolution | m/z 2120.9744 [M + Na]⁺ |
| Mass Spectrum | |
| HPLC Analysis | 95.4% (area %), SunFire C18 Column, |
| | Detector @ 230 nm |
| HPLC Analysis | >99% (area %), SunFire C18 Column, ELS |
| | Detector |

Conditions for LCMS and HPLC Purity Tests

LCMS:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 34.00 | 5 | 95 |
| 34.10 | 85 | 15 |
| 39.00 | 85 | 15 |

Detection: 230 nm, MS with turbo ion spray ionization

HPLC Purity:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 33.00 | 5 | 95 |
| 34.00 | 85 | 15 |
| 37.00 | 85 | 15 |

Detection: Photodiode array from 190 nm-370 nm (extraction at 230 nm)

ELSD, 120° C., 3.0 L/min nitrogen

TABLE 2
¹H and ¹³C NMR Assignments for Aglycone
region of C-11 Hydroxy Avicin D

| Position | ¹³C | ¹H |
|---|---|---|
| 1 | 41.9 | 1.40 m |
| | | 2.00 m |
| 2 | 27.5 | 1.65 m |
| | | 1.82 m |
| 3 | 88.9 | 3.37 m |
| 4 | 40.6 | — |
| 5 | 57.2 | 0.84 d, 12 Hz |
| 6 | 19.8 | 1.33 m |
| | | 1.65 m |
| 7 | 35.3 | 1.31 m |
| | | 1.55 m |
| 8 | 44.7 | — |
| 9 | 55.9 | 1.75 m |
| 10 | 39.3 | — |
| 11 | 68.2 | 4.20 m |
| 12 | 127.7 | 5.32-5.35 m |
| 13 | 147.1 | — |
| 14 | 42.8 | — |
| 15 | 36.3 | 1.55 m |
| 16 | 74.2 | 4.42-4.49 m |
| 17 | 52.1 | — |
| 18 | 41.0 | 2.98 dd, 4, 14 Hz |
| 19 | 48.4 | 1.27 m |
| | | 2.50 m |
| 20 | 35.9 | — |
| 21 | 78.7 | 5.51 dd, 5.5, 11 Hz |
| 22 | 36.4 | 1.75 m |
| | | 2.18 dd, 5, 13 Hz |
| 23 | 28.6 | 0.99 s |
| 24 | 17.2 | 0.78 s |
| 25 | 18.4 | 1.05 s |
| 26 | 18.8 | 0.79 s |
| 27 | 23.3 | 1.55 s |
| 28 | 175.4 | — |
| 29 | 29.5 | 0.88 s |
| 30 | 19.6 | 1.07 s |

TABLE 3
¹H and ¹³C NMR Assignments for C-3 Glycoside
region of C-11 Hydroxy Avicin D

| Position | ¹³C | ¹H |
|---|---|---|
| GN-1 | 104.7 | 4.42-4.49 m |
| GN-2 | 58.0 | 3.59-3.72 m |
| GN-3 | 75.9 | 3.45 t, 9.5 Hz |
| GN-4 | 72.5 | 3.20-3.30 m |
| GN-5 | 77.5 | 3.48-3.59 m |
| GN-6 | 69.8 | 3.70-3.80 m |
| GN-7 | 173.5 | 4.03-4.09 m |
| GN-8 | 23.3 | 1.95 s |
| Fuc-1 | 104.2 | 4.58 t, 7.5 Hz |
| Fuc-2 | 82.5 | 3.59-3.72 m |
| Fuc-3 | 74.7 | 3.59-3.72 m |
| Fuc-4 | 73.0 | 3.59-3.72 m |
| Fuc-5 | 71.9 | 3.59-3.72 |
| Fuc-6 | 16.9 | 1.25-1.30 m |
| Xyl-1 | 107.4 | 4.42-4.49 m |
| Xyl-2 | 76.4 | 3.32-3.41 m |
| Xyl-3 | 77.8 | 3.32-3.41 m |
| Xyl-4 | 71.3 | 3.48-3.59 m |
| Xyl-5 | 67.3 | 3.20-3.30 m |
| | | 3.97 dd, 5.5 & 11.5 Hz |

TABLE 4
¹H and ¹³C NMR Assignments for C-28 Glycoside
region of C-11 Hydroxy Avicin D

| Position | ¹³C | ¹H |
|---|---|---|
| G₁-1 | 95.5 | 5.31-5.35 m |
| G₁-2 | 76.4 | 3.48-3.59 m |
| G₁-3 | 78.3 | 3.32-3.41 m |
| G₁-4 | 71.3 | 3.32-3.41 |
| G₁-5 | 78.7 | 3.20-3.30 |
| G₁-6 | 62.3 | 3.60-3.70 m |
| | | 3.70-3.80 m |
| Rha-1 | 101.4 | 5.31-5.35 m |
| Rha-2 | 71.4 | 4.18-4.21 m |
| Rha-3 | 82.7 | 3.83-3.90 m |
| Rha-4 | 78.7 | 3.59-3.72 m |
| Rha-5 | 69.2 | 3.83-3.90 m |
| Rha-6 | 18.8 | 1.33 d, 6.5 Hz |
| G2-1 | 105.9 | 4.42-4.49 m |
| G2-2 | 75.4 | 3.20-3.30 m |
| G2-3 | 79.2 | 3.32-3.41 m |
| G2-4 | 71.6 | 3.32-3.41 m |
| G2-5 | 77.8 | 3.32-3.41 m |
| G2-6 | 62.5 | 3.72-3.80 m |
| | | 3.80-3.84 m |
| Ara-1 | 111.2 | 5.31-5.35 m |
| Ara-2 | 84.0 | 4.03-4.09 m |
| Ara-3 | 78.8 | 3.83-3.90 m |
| Ara-4 | 85.8 | 4.03-4.09 m |
| Ara-5 | 63.2 | 3.59-3.72 m |
| | | 3.70-3.80 m |

TABLE 5
¹H and ¹³C NMR Assignments for C-21 Monoterpene-Glycoside
region of C-11 Hydroxy Avicin D

| Position | ¹³C | ¹H |
|---|---|---|
| MT₁-1 | 168.8 | — |
| MT₁-2 | 132.6 | — |
| MT₁-3 | 148.2 | 6.89-6.96 m |
| MT₁-4 | 24.4 | 2.30-2.55 m |
| MT₁-5 | 41.5 | 1.72-1.80 m |
| MT₁-6 | 81.2 | — |
| MT₁-7 | 144.1 | 5.96 dd, 11 & 18 Hz |
| MT₁-8 | 116.2 | 5.21-5.31 m |
| MT₁-9 | 56.6 | 4.33 s |
| MT₁-10 | 23.9 | 1.39 s |
| Qui-1 | 99.5 | 4.42-4.49 m |
| Qui-2 | 75.7 | 3.48-3.59 m |
| Qui-3 | 75.6 | 3.20-3.30 m |
| Qui-4 | 77.7 | 4.64 t, 9.5 Hz |
| Qui-5 | 71.0 | 3.48-3.49 m |
| Qui-6 | 18.4 | 1.13 d, 6 Hz |
| MT₂-1 | 168.3 | — |
| MT₂-2 | 133.0 | — |
| MT₂-3 | 148.6 | 6.89-6.96 m |
| MT₂-4 | 24.6 | 2.30-2.55 m |
| MT₂-5 | 42.1 | 1.60-1.70 m |
| MT₂-6 | 73.8 | — |
| MT₂-7 | 146.0 | 5.92 dd, 10.5 & 17.5 Hz |
| MT₂-8 | 112.7 | 5.06 dd, 1.5 & 11 Hz |
| | | 5.21-5.28 m |
| MT₂-9 | 56.8 | 4.33 s |
| MT₂-10 | 28.1 | 1.27 s |

TABLE 6
Overview of Analysis of Oxidatively Rearranged Avicin D

| TEST | RESULT/REFERENCE |
|---|---|
| Appearance | White solid |
| 500 MHz ¹H NMR | Assignments and spectra - Agree with structure |
| Spectrum (CD₃OD) | |
| 500 MHz ¹³C | Assignments and spectra - Agree with structure |
| NMR Spectrum | |
| LC-MS (TIS) | m/z 2120 [M + Na]⁺, Agrees with structure |
| Analysis | |
| High Resolution | m/z 2118.9458 [M + Na]⁺, Agrees with |
| Mass Spectrum | structure |
| HPLC Analysis | >99% (area %), SunFire C18 Column, Detector |
| | @ 230 nm |
| HPLC Analysis | >99% (area %), SunFire C18 Column, ELS |
| | Detector |
| Elemental Analysis | Elemental Analysis consistent with formula |
| | C₉₈H₁₅₃NO₄₇Na |

Conditions for LCMS and HPLC Purity Tests

LCMS:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 34.00 | 5 | 95 |
| 34.10 | 85 | 15 |
| 39.00 | 85 | 15 |

Detection: 230 nm, MS with turbo ion spray ionization

HPLC Purity:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 33.00 | 5 | 95 |
| 34.00 | 85 | 15 |
| 37.00 | 85 | 15 |

Detection: Photodiode array from 190 nm-370 nm (extraction at 230 nm)

ELSD, 120 ° C., 3.0 L/min nitrogen.

TABLE 7
¹H and ¹³C NMR Assignments for Aglycone
region of Oxidatively Rearranged Avicin D

| Position | ¹³C | ¹H |
|---|---|---|
| 1 | 39.9 | 1.37 m |
| | | 1.76 m |
| 2 | 27.0 | 1.76 m |
| | | 1.91 m |
| 3 | 89.1 | 3.37 |
| 4 | 40.2 | — |
| 5 | 56.9 | 0.82 m |
| 6 | 20.4 | 1.49 d, 13 Hz |
| | | 1.71 m |
| 7 | 42.6 | 1.28 m |
| | | 2.17 d, 12.5 Hz |
| 8 | 41.5 | — |
| 9 | 54.6 | 1.30 m |
| 10 | 37.7 | — |
| 11 | 52.8 | 3.15 t, 5 Hz |
| 12 | 63.3 | 3.27 m |
| 13 | 39.3 | — |
| 14 | 163.5 | — |
| 15 | 122.5 | 5.89 d, 6.5 Hz |
| 16 | 72.5 | 4.49 m |
| 17 | 53.9 | — |
| 18 | 42.7 | 2.91 dd, 4.8, 13 Hz |
| 19 | 41.2 | 1.56 m, |
| | | 2.11 m |
| 20 | 35.8 | — |
| 21 | 78.5 | 5.16 dd, 4.5, 10 Hz |
| 22 | 35.7 | 1.90 m, |
| | | 2.25 dd, 4, 14 Hz |
| 23 | 28.2 | 0.96 |
| 24 | 16.9 | 0.79 |
| 25 | 18.1 | 1.08 s |
| 26 | 28.5 | 0.94 s |
| 27 | 27.3 | 1.14 s |
| 28 | 175.8 | — |
| 29 | 29.9 | 0.98 s |
| 30 | 19.9 | 1.02 s |

TABLE 8
¹H and ¹³C NMR Assignments for C-3 Glycoside
region of Oxidatively Rearranged Avicin D

| Position | ¹³C | ¹H |
|---|---|---|
| GN-1 | 104.7 | 4.44 m |
| GN-2 | 57.9 | 3.64 m |
| GN-3 | 75.9 | 3.44 m |
| GN-4 | 71.2 | 3.24 m |
| GN-5 | 77.6 | 3.48 m |
| GN-6 | 70.1 | 3.74 m |
| | | 4.11 d, 12 Hz |
| GN-7 | 173.5 | — |
| GN-8 | 23.3 | 1.95 s |
| Fuc-1 | 104.4 | 4.54 d, 7 Hz |
| Fuc-2 | 82.1 | 3.64 m |
| Fuc-3 | 74.9 | 3.71 m |
| Fuc-4 | 72.8 | 3.64 m |
| Fuc-5 | 71.9 | 3.59 m |
| Fuc-6 | 16.9 | 1.28 d, 5.5 Hz |
| Xyl-1 | 107.0 | 4.47 d, 7 Hz |
| Xyl-2 | 75.6 | 3.27 m |
| Xyl-3 | 77.8 | 3.34 m |
| Xyl-4 | 71.0 | 3.47 m |
| Xyl-5 | 67.4 | 3.26 m |
| | | 3.96 m |

TABLE 9
¹H and ¹³C NMR Assignments for C-28 Glycoside
region of Oxidatively Rearranged Avicin D

| Position | ¹³C | ¹H |
|---|---|---|
| G₁-1 | 95.9 | 5.18 d, 8 Hz |
| G₁-2 | 76.0 | 3.52 m |
| G₁-3 | 78.4 | 3.37 m |
| G₁-4 | 71.2 | 3.37 m |
| G₁-5 | 78.7 | 3.25 m |
| G₁-6 | 62.1 | 3.65 m |
| | | 3.73 m |
| Rha-1 | 101.3 | 5.35 bs |
| Rha-2 | 71.2 | 4.25 bs |
| Rha-3 | 83.1 | 3.96 m |
| Rha-4 | 78.9 | 3.68 m |
| Rha-5 | 69.1 | 3.95 m |
| Rha-6 | 18.7 | 1.37 m |
| G₂-1 | 106.1 | 4.52 d, 7.5 Hz |
| G₂-2 | 75.4 | 3.30 m |
| G₂-3 | 79.2 | 3.48 m |
| G₂-4 | 71.2 | 3.37 m |
| G₂-5 | 77.9 | 3.30 m |
| G₂-6 | 62.5 | 3.73 m |
| | | 3.83 m |
| Ara-1 | 111.2 | 5.37 bs |
| Ara-2 | 84.3 | 4.08 m |
| Ara-3 | 78.9 | 3.85 m |
| Ara-4 | 85.5 | 4.02 m |
| Ara-5 | 63.2 | 3.63 m |
| | | 3.74 m |

TABLE 10
¹H and ¹³C NMR Assignments for C-21 Monoterpene-Glycoside
region of Oxidatively Rearranged Avicin D

| Position | ¹³C | ¹H |
|---|---|---|
| MT₁-1 | 168.8 | — |
| MT₁-2 | 132.8 | — |
| MT₁-3 | 148.7 | 6.93 t, 7.5 Hz |
| MT₁-4 | 24.5 | 2.45 m |
| MT₁-5 | 41.4 | 1.75 m |
| MT₁-6 | 81.1 | — |
| MT₁-7 | 144.2 | 5.96 dd, 11, 17.5 Hz |
| MT₁-8 | 116.1 | 5.22 m |
| | | 5.30 d, 18 Hz |
| MT₁-9 | 56.7 | 4.32 s |
| MT₁-10 | 23.9 | 1.39 s |
| Qui-1 | 99.5 | 4.42 d, 8 Hz |
| Qui-2 | 75.7 | 3.26 m |
| Qui-3 | 76.2 | 3.54 m |
| Qui-4 | 77.8 | 4.64 t, 9.5 Hz |
| Qui-5 | 71.0 | 3.49 m |
| Qui-6 | 18.4 | 1.12 d, 6.5 Hz |
| MT₂-1 | 168.3 | — |
| MT₂-2 | 132.6 | — |
| MT₂-3 | 148.6 | 6.95 t, 7.5 Hz |
| MT₂-4 | 24.6 | 2.37 m |
| MT₂-5 | 42.1 | 1.64 m |
| MT₂-6 | 73.8 | — |
| MT₂-7 | 146.1 | 5.92 dd, 11, 17.5 Hz |
| MT₂-8 | 112.7 | 5.06 d, 10.5 Hz |
| | | 5.22 m |
| MT₂-9 | 56.9 | 4.32 s |
| MT₂-10 | 28.1 | 1.27 s |

TABLE 11
¹H and ¹³C chemical shift assignment for the aglycone
region of Oxidatively Rearranged Avicin D (Avicin D as a
comparison compound 2^{nd} and 3^{rd} Columns; Oxidatively
Rearranged Avicin D 4^{th} and 5^{th} Columns)

| Position | ¹³C | ¹H | ¹³C | ¹H |
|---|---|---|---|---|
| 1 | 39.9 | 1.07 m | 39.6 | 1.37 m |
| | | 1.62 m | | 1.76 m |
| 2 | 27.1 | 1.66 m | 26.6 | 1.75 m |
| | | 1.88 m | | 1.91 m |
| 3 | 89.8 | 3.25 m | 88.6 | 3.36 m |
| 4 | 40.1 | — | 39.7 | — |
| 5 | 57.0 | 0.80 m | 56.5 | 0.82 m |
| 6 | 19.5 | 1.40 m | 20.1 | 1.49 d (13.0) |
| | | 1.62 m | | 1.71 m |
| 7 | 34.6 | 1.42 m | 42.2 | 1.28 m |
| | | 1.59 m | | 2.17 d (12.6) |
| 8 | 40.8 | — | −42 | — |
| 9 | 48.1 | 1.69 m | 54.3 | 1.30 d (5.9) |
| 10 | 37.9 | — | 37.1 | — |
| 11 | 24.5 | 1.92 m | 52.4 | 3.15 t (5.2) |
| 12 | 124.0 | 5.35 m | 62.9 | 3.26 m |
| 13 | 143.6 | — | 38.7 | — |
| 14 | 42.6 | — | 162.9 | — |
| 15 | 36.1 | 1.52 t (13.3) | 122.1 | 5.89 d (7.0) |
| | | 1.60 m | | |
| 16 | 74.2 | 4.49 m | 72.1 | 4.49 d (7.0) |
| 17 | 52.3 | — | | — |
| 18 | 41.6 | 2.97 dd (4.4, 13.7) | 42.3 | 2.91 dd (4.8. 13.7) |
| 19 | 48.7 | 1.18 dd (4.4, 13.3) | 40.8 | 1.56 dd (4.8, 13.7) |
| | | 2.52 t (13.7) | | 2.11 t (13.7) |
| 20 | 35.9 | — | 35.1 | — |
| 21 | 78.6 | 5.49 dd (5.6, 11.1) | 78.1 | 5.16 dd (4.8, 10.7) |
| 22 | 36.4 | 1.72 m | 35.2 | 1.88 dd (10.7, 14.4) |
| | | 2.15 dd (5.6, 13.7) | | 2.22 dd (4.8, 14.4) |
| 23 | 28.6 | 0.98 s | 27.9 | 0.96 s |
| 24 | 17.1 | 0.77 s | 16.4 | 0.79 s |
| 25 | 16.2 | 0.95 s | 17.7 | 1.07 s |
| 26 | 17.7 | 0.77 s | 28.1 | 0.94 s |
| 27 | 27.4 | 1.43 s | 26.9 | 1.14 s |
| 28 | 175.3 | — | | — |
| 29 | 29.4 | 0.87 s | 29.5 | 0.98 s |
| 30 | 19.4 | 1.04 s | 19.4 | 1.02 s |

TABLE 12
¹H and ¹³C chemical shift assignment for the C-3 glycoside
region of Oxidatively Rearranged Avicin D (Avicin D as a
comparison compound 2^{nd} and 3^{rd} Columns; Oxidatively
Rearranged Avicin D 4^{th} and 5^{th} Columns)

| Position | ¹³C | ¹H | ¹³C | ¹H |
|---|---|---|---|---|
| GlcNAc-1 | 104.8 | 4.44 d (8.2) | 104.3 | 4.44 d (8.2) |
| GlcNAc-2 | 57.9 | 3.64 m | 57.6 | 3.64 m |
| GlcNAc-3 | 75.7 | 3.43 m | 75.5 | 3.44 m |
| GlcNAc-4 | 72.2 | 3.29 m | 72.1 | 3.24 m |
| GlcNAc-5 | 77.1 | 3.45 m | 77.2 | 3.48 m |
| GlcNAc-6 | 69.9 | 3.76 m | 69.7 | 3.74 m |
| | | 4.07 dd (1.5, 11.5) | | 4.10 d (11.5) |
| GlcNAc—C═O | 173.4 | — | 173.1 | — |
| GlcNAc—Me | 23.2 | 1.94 s | 22.8 | 1.94 s |
| Fuc-1 | 103.8 | 4.51 m | 103.7 | 4.54 d (7.8) |
| Fuc-2 | 82.3 | 3.62 m | 81.7 | 3.64 m |
| Fuc-3 | 75.0 | 3.62 m | 74.5 | 3.71 m |
| Fuc-4 | 72.6 | 3.66 m | 72.5 | 3.64 m |
| Fuc-5 | 71.7 | 3.59 q (6.7) | 71.6 | 3.59 m |
| Fuc-6 | 16.8 | 1.27 d (6.3) | 16.5 | 1.27 d (5.9) |
| Xyl-1 | 106.9 | 4.47 d (7.8) | 106.6 | 4.47 d (7.4) |
| Xyl-2 | 76.0 | 3.30 m | 75.4 | 3.27 m |
| Xyl-3 | 77.5 | 3.33 m | 77.3 | 3.34 m |
| Xyl-4 | 71.1 | 3.49 m | 70.8 | 3.47 m |
| Xyl-5 | 67.3 | 3.25 m | 67.0 | 3.26 m |
| | | 3.97 dd (5.6, 11.5) | | 3.96 m |

TABLE 13
¹H and ¹³C chemical shift assignment for the C-28 glycoside
region of Oxidatively Rearranged Avicin D (Avicin D as a
comparison compound 2^{nd} and 3^{rd} Columns; Oxidatively
Rearranged Avicin D 4^{th} and 5^{th} Columns)

| Position | ¹³C | ¹H | ¹³C | ¹H |
|---|---|---|---|---|
| Glc-1 | 95.3 | 5.32 m | 95.6 | 5.18 d (7.8) |
| Glc-2 | 76.4 | 3.52 m | 75.5 | 3.52 m |
| Glc-3 | 78.1 | 3.36 m | 78.0 | 3.37 m |
| Glc-4 | 71.1 | 3.36 m | 70.9 | 3.37 m |
| Glc-5 | 78.6 | 3.30 m | 78.2 | 3.25 m |
| Glc-6 | 62.2 | 3.64 m | 61.8 | 3.65 m |
| | | 3.79 dd (2.2, 10.4) | | 3.73 m |
| Rha-1 | 101.3 | 5.32 m | 100.9 | 5.35 bs |
| Rha-2 | 71.5 | 4.21 dd (1.9, 3.0) | 70.9 | 4.24 dd (1.9, 3.3) |
| Rha-3 | 82.6 | 3.88 m | 82.8 | 3.96 m |
| Rha-4 | 78.6 | 3.66 m | 78.6 | 3.68 m |
| Rha-5 | 69.1 | 3.87 m | 68.6 | 3.95 m |
| Rha-6 | 18.6 | 1.33 d (6.3) | 18.3 | 1.37 d (6.3) |
| Glc′-1 | 105.8 | 4.49 d (7.8) | 105.6 | 4.52 d (7.8) |
| Glc′-2 | 75.3 | 3.27 m | 75.1 | 3.30 m |
| Glc′-3 | 79.0 | 3.52 m | 78.7 | 3.48 m |
| Glc′-4 | 71.2 | 3.36 m | 70.9 | 3.37 m |
| Glc′-5 | 77.7 | 3.27 m | 77.6 | 3.30 m |
| Glc′-6 | 62.3 | 3.72 dd (4.4, 11.8) | 62.2 | 3.73 |
| | | 3.82 dd (2.2, 11.8) | | 3.83 dd (1.5, 9.6) |
| Ara-1 | 111.0 | 5.35 m | 110.7 | 5.36 bs |
| Ara-2 | 83.9 | 4.09 dd (1.9, 3.7) | 83.9 | 4.08 dd (1.5, 3.7) |
| Ara-3 | 78.6 | 3.88 m | 78.5 | 3.85 dd (3.7, 6.6) |
| Ara-4 | 85.5 | 4.03 ddd (3.3, 5.2, 6.3) | 85.1 | 4.02 ddd (3.3, |
| | | | | 5.2, 6.6) |
| Ara-5 | 63.1 | 3.62 m | 62.7 | 3.63 m |
| | | 3.75 m | | 3.74 m |

TABLE 14
¹H and ¹³C chemical shift assignment for the C-28 glycoside
region of Oxidatively Rearranged Avicin D (Avicin D as a
comparison compound 2^{nd} and 3^{rd} Columns; Oxidatively
Rearranged Avicin D 4^{th} and 5^{th} Columns)

| Position | ¹³C | ¹H | ¹³C | ¹H |
|---|---|---|---|---|
| MT₁-1 | 168.7 | — | 168.1 | — |
| MT₁-2 | 132.9 | — | 132.3 | — |
| MT₁-3 | 148.0 | 6.90 t (7.8) | 148.2 | 6.92 t (7.8) |
| MT₁-4 | 24.3 | 2.44 m | 24.1 | 2.45 m |
| MT₁-5 | 41.3 | 1.75 m | 41.0 | 1.75 m |
| MT₁-6 | 81.0 | — | 80.6 | — |
| MT₁-7 | 144.0 | 5.95 dd (10.7, 17.4) | 143.8 | 5.95 dd (11.1, 17.8) |
| MT₁-8 | 116.0 | 5.22 dd (1.1, 9.6) | 115.7 | 5.22 d (9.6) |
| | | 5.30 dd (1.1, 17.4) | | 5.30 d (17.8) |
| MT₁-9 | 56.6 | 4.32 s | 56.3 | 4.32 s |
| MT₁-10 | 23.8 | 1.38 s | 23.6 | 1.39 s |
| Qui-1 | 99.3 | 4.42 d (7.8) | 99.1 | 4.42 d (8.2) |
| Qui-2 | 75.5 | 3.27 m | 75.4 | 3.26 m |
| Qui-3 | 75.6 | 3.54 t (9.6) | 75.5 | 3.54 t (9.3) |
| Qui-4 | 77.6 | 4.64 t (9.6) | 77.4 | 4.64 t (9.6) |
| Qui-5 | 70.9 | 3.49 m | 70.7 | 3.49 m |
| Qui-6 | 18.3 | 1.12 d (6.3) | 18.0 | 1.12 d (6.3) |
| MT₂-1 | 168.2 | — | 168.1 | — |
| MT₂-2 | 132.5 | — | 132.3 | — |
| MT₂-3 | 148.5 | 6.94 t (7.8) | 148.2 | 6.94 t (7.8) |
| MT₂-4 | 24.5 | 2.37 m | 24.2 | 2.37 m |
| MT₂-5 | 41.9 | 1.64 m | 41.7 | 1.64 m |
| MT₂-6 | 73.6 | — | 73.1 | — |
| MT₂-7 | 145.9 | 5.91 dd (10.7, 17.4) | 145.6 | 5.91 dd (10.7, 17.4) |
| MT₂-8 | 112.5 | 5.05 dd (1.5, 10.7) | 112.3 | 5.05 dd (1.5, 10.7) |
| | | 5.22 dd (1.5, 17.4) | | 5.22 dd (1.5, 17.4) |
| MT₂-9 | 56.5 | 4.32 s | 56.3 | 4.32 s |
| MT₂-10 | 27.9 | 1.27 s | 27.7 | 1.27 s |

### Example 2

### Preparation of a C-11 Ketone Derivative of Avicin D

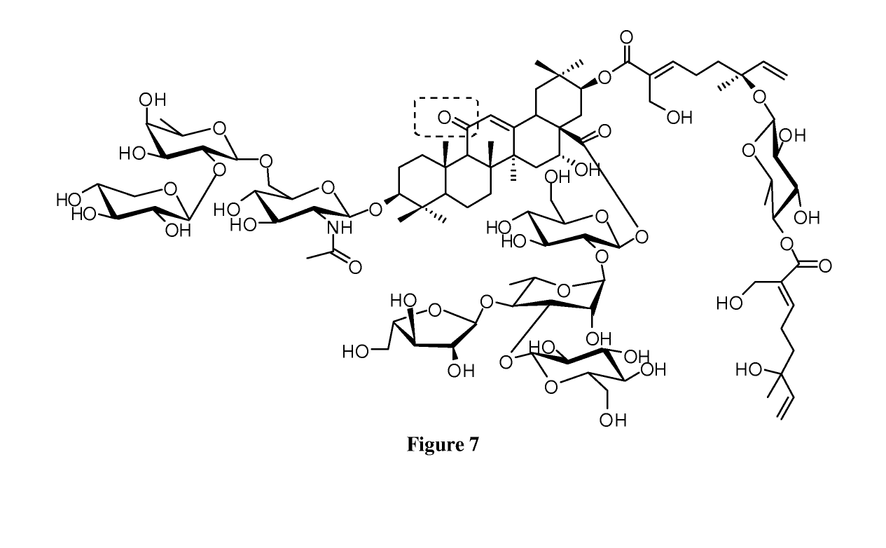

C-11 Keto Avicin D [ALB-153384] corresponds to the formula shown in FIG. 7 and the following chemical name:
(3S,4aR,5R,6aS,6bR,10S,12aS)-((2S,3S,4S,5S)-3-((2S,3R,4S,5S)-5-((2S,3S,5S)-3,4-dihydroxy-5-(hydroxymethyl)tetrahydrofuran-2-yloxy)-3-hydroxy-6-methyl-4-((2S,3S,4S,5S)-3,4,5-trihydroxy-6-(hydroxymethyl)tetrahydro-2H-pyran-2-yloxy)tetrahydro-2H-pyran-2-yloxy)-4,5-dihydroxy-6-(hydroxymethyl)tetrahydro-2H-pyran-2-yl) 10-((2R,3S,4R,5S)-3-acetamido-6-(((2R,3S,4S,5R)-4,5-dihydroxy-6-methyl-3-(2S,3S,4S,5R)-3,4,5-trihydroxytetrahydro-2H-pyran-2-yloxy)tetrahydro-2H-pyran-2-yloxy)methyl)-4,5-dihydroxytetrahydro-2H-pyran-2-yloxy)-3-((6S,E)-6-((2S,3S,4R,5S)-3,4-dihydroxy-5-(E)-6-hydroxy-2-(hydroxymethyl)-6-methylocta-2,7-dienoyloxy)-6-methyltetrahydro-2H-pyran-2-yloxy)-2-(hydroxymethyl)-6-methylocta-2,7-dienoyloxy)-5-hydroxy-2,2,6a,6b,9,9,12a-heptamethyl-13-oxo-1,2,3,4,4a,5,6,6a,6b,7,8,8a,9,10,11,12,12a,12b,13,14b-icosahydropicene-4a-carboxylate.
   This compound was synthesized as follows:

To a solution of Avicin D (0.25 g, 0.12 mmol) and laccase enzyme (L-Y 120, 50 mg) in 50 mM sodium citrate buffer (pH 5.5, 75 mL) was added HOBt (68 mg, 0.44 mmol) in methanol (10 mL). The reaction mixture was incubated at 27° C., 100 rpm agitation for 7 h. The reaction mixture was diluted with water (200 mL) and loaded onto a pre-conditioned Alltech C18 SPE cartridge (10 g). The cartridge was washed with water (300 mL) and the product was eluted with methanol (150 mL). The methanol elute was concentrated under reduced pressure to ca. 10 mL volume and the C-11 ketone derivative was purified by preparative HPLC.

Column—Waters SunFire C18 OBD (150×50 mm, 5 ρm)

Column temperature—Ambient

Solvents—A (water+0.1% formic acid); B (acetonitrile+0.1% formic acid)

Gradient—Linear (25% B to 30% B within 30 min).

Flowrate—100 mL/min and λ220 nm

Pure fractions were combined and lyophilized to afford the desired C-11 Keto Avicin D (41 mg, 16%) as a white solid.

TABLE 15
Overview of Analysis of C-11 Keto Avicin D

| TEST | RESULT/REFERENCE |
|---|---|
| Appearance | White solid |
| 500 MHz ¹H NMR | Assignments and spectra -Agree with structure |
| Spectrum (CD₃OD) | |
| 500 MHz ¹³C | Assignments and spectra -Agree with structure |
| NMR Spectrum | |
| LC-MS (TIS) | m/z 2120 [M + Na]⁺, Agrees with structure |
| Analysis | |
| High Resolution | m/z 2118.9612 [M + Na]⁺, Agrees with |
| Mass Spectrum | structure |
| HPLC Analysis | >99% (area %), SunFire C18 Column, Detector |
| | @ 230 nm |
| HPLC Analysis | >99% (area %), SunFire C18 Column, ELS |
| | Detector |

Conditions for LCMS and HPLC Purity Tests

LCMS:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 34.00 | 5 | 95 |
| 34.10 | 85 | 15 |
| 39.00 | 85 | 15 |

Detection: 230 nm, MS with turbo ion spray ionization

HPLC Purity:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 33.00 | 5 | 95 |
| 34.00 | 85 | 15 |
| 37.00 | 85 | 15 |

Detection: Photodiode array from 190 nm-370 nm (extraction at 230 nm)

ELSD, 120° C., 3.0 L/min nitrogen

TABLE 16
¹H and ¹³C NMR Assignments for Aglycone
region of C-11 Keto Avicin D

| Position | ¹³C | ¹H |
|---|---|---|
| 1 | 40.5 | 2.67 (d, 13 Hz) |
| | | 1.12-1.13 m |
| 2 | 27.3 | 1.60-1.80 m |
| | | 1.22-1.48 m |
| 3 | 89.1 | 3.24-3.40 m |
| 4 | 40.6 | — |
| 5 | 56.8 | 0.74 m |
| 6 | 18.7 | 1.22-1.48 m |
| 7 | 34.8 | 1.60-1.80 m |
| | | 1.22-1.48 m |
| 8 | 47.1 | — |
| 9 | 62.3 | 2.54-2.60 m |
| 10 | 38.4 | — |
| 11 | 203.2 | — |
| 12 | 128.6 | 5.63 s |
| 13 | 171.8 | — |
| 14 | 44.8 | — |
| 15 | 36.1 | 1.60-1.80 m |
| 16 | 73.4 | 4.42-4.57 m |
| 17 | 52.4 | — |
| 18 | 42.3 | 3.08 (dd, 3.5 Hz, 13.5 Hz) |
| 19 | 47.3 | 1.22-1.48 m |
| | | 2.55-2.60 m |
| 20 | 35.9 | — |
| 21 | 78.2 | 5.52 (dd, 5.5 Hz, 11 Hz) |
| 22 | 35.8 | 1.60-1.85 m |
| | | 2.25 (dd, 5 Hz, 13.5 Hz) |
| 23 | 28.7 | 0.99 s |
| 24 | 17.2 | 0.78 s |
| 25 | 17.3 | 1.12-1.13 m |
| 26 | 19.6 | 0.94 s |
| 27 | 24.6 | 1.60-1.85 m |
| 28 | 175.2 | — |
| 29 | 29.3 | 0.91 s |
| 30 | 19.4 | 1.10 s |

TABLE 17
¹H and ¹³C NMR Assignments for C-3 Glycoside
region of C-11 Keto Avicin D

| Position | ¹³C | ¹H |
|---|---|---|
| Glc″-1 | 104.7 | 4.45 (d, 8 Hz) |
| Glc″-2 | 58.1 | 3.61 m |
| Glc″-3 | 75.8 | 3.54 m |
| Glc″-4 | 72.4 | 3.29 m |
| Glc″-5 | 77.4 | 3.46 m |
| Glc″-6 | 69.9 | 3.74 m, 4.09 m |
| GlcNAc—C═O | 173.5 | — |
| GlcNAc—Me | 23.3 | 1.94 s |
| Fuc-1 | 104.0 | 4.48 m |
| Fuc-2 | 82.8 | 3.61 m |
| Fuc-3 | 75.1 | 3.61 m |
| Fuc-4 | 72.8 | 3.61 m |
| Fuc-5 | 71.8 | 3.57 m |
| Fuc-6 | 16.9 | 1.28 (d, 6 Hz) |
| Xyl-1 | 107.3 | 4.46 (d, 7.5 Hz) |
| Xyl-2 | 75.8 | 3.26 m |
| Xyl-3 | 77.6 | 3.31 m |
| Xyl-4 | 71.2 | 3.49 m |
| Xyl-5 | 67.4 | 3.25 m , 3.98 (dd, 5 Hz, 11.5 Hz) |

TABLE 18
¹H and ¹³C NMR Assignments for C-28 Glycoside
region of C-11 Keto Avicin D

| Position | ¹³C | ¹H |
|---|---|---|
| Glc-1 | 95.6 | 5.28 m |
| Glc-2 | 76.4 | 3.49 m |
| Glc-3 | 78.3 | 3.36 m |
| Glc-4 | 71.2 | 3.37 m |
| Glc-5 | 78.8 | 3.30 m |
| Glc-6 | 62.5 | 3.69 m |
| Rha-1 | 101.4 | 5.28 m |
| Rha-2 | 71.3 | 4.21 m |
| Rha-3 | 82.8 | 3.85 m |
| Rha-4 | 78.8 | 3.66 m |
| Rha-5 | 69.3 | 3.85 m |
| Rha-6 | 18.8 | 1.34 (d, 6 Hz) |
| Glc′-1 | 105.9 | 4.53 m |
| Glc′-2 | 75.4 | 3.28 m |
| Glc′-3 | 79.1 | 3.53 m |
| Glc′-4 | 71.5 | 3.38 m |
| Glc′-5 | 77.9 | 3.26 m |
| Glc′-6 | 62.5 | 3.74 m, 3.82 m |
| Ara-1 | 111.2 | 5.28 m |
| Ara-2 | 85.9 | 4.09 m |
| Ara-3 | 78.8 | 3.89 m |
| Ara-4 | 84.0 | 4.07 m |
| Ara-5 | 63.2 | 3.66 m, 3.74 m |

TABLE 19
¹H and ¹³C NMR Assignments for C-21 Monoterpene-Glycoside
region of C-11 Keto Avicin D

| Position | ¹³C | ¹H |
|---|---|---|
| MT₁-1 | 168.7 | — |
| MT₁-2 | 132.6 | — |
| MT₁-3 | 148.6 | 6.90-6.96 m |
| MT₁-4 | 24.5 | 2.45 m |
| MT₁-5 | 41.5 | 1.75 m |
| MT₁-6 | 81.1 | — |
| MT₁-7 | 144.1 | 5.89-5.99 m |
| MT₁-8 | 116.2 | 5.28 m |
| MT₁-9 | 56.6 | 4.33 s |
| MT₁-10 | 23.9 | 1.39 s |
| Qui-1 | 99.5 | 4.43 (d, 8 Hz) |
| Qui-2 | 75.6 | 3.28 m |
| Qui-3 | 75.7 | 3.55 m |
| Qui-4 | 77.8 | 4.64 (t, 9.5 Hz) |
| Qui-5 | 71.0 | 3.49 m |
| Qui-6 | 18.7 | 1.12 (d, 6 Hz) |
| MT₂-1 | 168.3 | — |
| MT₂-2 | 132.9 | — |
| MT₂-3 | 148.4 | 6.90-6.96 m |
| MT₂-4 | 24.6 | 2.38 m |
| MT₂-5 | 42.1 | 1.65 m |
| MT₂-6 | 73.8 | — |
| MT₂-7 | 146.0 | 5.88-5.99 m |
| MT₂-8 | 112.7 | 5.28 m |
| MT₂-9 | 56.7 | 4.38 s |
| MT₂-10 | 28.1 | 1.28 s |

### Example 3

### Preparation of a Hydroperoxy Derivative of Avicin D

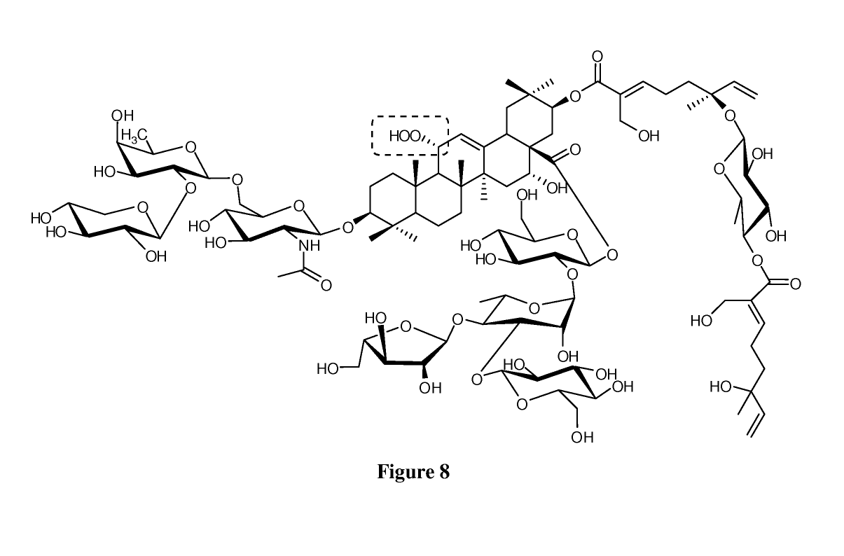

C-11 Hydroperoxy Avicin D [ALB-154737] corresponds to the formula shown in FIG. 8 and the following chemical name:
(3S,4aR,5R,6aS,6bR,10S,12aS,13R,14bS)-((2S,3S,4S,5S)-3-((2S,3R,4S,5S)-5-((2S,3S,5S)-3,4-dihydroxy-5-(hydroxymethyl)tetrahydrofuran-2-yloxy)-3-hydroxy-6-methyl-4-((2S,3S,4S,5S)-3,4,5-trihydroxy-6-(hydroxymethyl)tetrahydro-2H-pyran-2-yloxy)tetrahydro-2H-pyran-2-yloxy)-4,5-dihydroxy-6-(hydroxymethyl)tetrahydro-2H-pyran-2-yl) 10-((2R,3S,4R,5S)-3-acetamido-6-(((2R,3S,4S,5R)-4,5-dihydroxy-6-methyl-3-((2S,3S,4S,5R)-3,4,5-trihydroxytetrahydro-2H-pyran-2-yloxy)tetrahydro-2H-pyran-2-yloxy)methyl)-4,5-dihydroxytetrahydro-2H-pyran-2-yloxy)-3-((6S,E)-6-((2S,3S,4R,5S)-3,4-dihydroxy-5-(E)-6-hydroxy-2-(hydroxymethyl)-6-methylocta-2,7-dienoyloxy)-6-methyltetrahydro-2H-pyran-2-yloxy)-2-(hydroxymethyl)-6-methylocta-2,7-dienoyloxy)-13-hydroperoxy-5-hydroxy-2,2,6a,6b,9,9,12a-heptamethyl-1,2,3,4,4a,5,6,6a,6b,7,8,8a,9,10,11,12,12a,12b,13,14b-icosahydropicene-4a-carboxylate
   This compound was synthesized as follows:

To a solution of Avicin D (0.15 g, 0.07 mmol) and laccase enzyme (L-RSL, 6.0 mL) in 50 mM sodium citrate buffer (pH 5.5, 60 mL) was added HOBt (40 mg, 0.26 mmol) in methanol (6.0 mL). The reaction mixture was incubated at 27 ° C., 100 rpm agitation for 21 h. The reaction mixture was diluted with water (200 mL) and loaded onto a pre-conditioned Alltech C18 SPE cartridge (10 g). The cartridge was washed with water (200 mL) and the product was eluted with methanol (200 mL). The methanol elute was concentrated under reduced pressure to ca. 10 mL volume and the C-11 hydroperoxy derivative was purified by preparative HPLC.

Column—Waters SunFire C18 OBD (150×50 mm, 5 μm)

Column temperature—Ambient

Solvents—A (water+0.1% formic acid); B (acetonitrile+0.1% formic acid)

Gradient—Linear (18% B to 33% B within 26 min).

Flowrate—100 mL/min and λ220 nm

Pure fractions were combined and lyophilized to afford C-11 Hydroperoxy Avicin D (83.3 mg, 54%) as a white solid.

TABLE 20
Overview of Analysis of C-11 Hydroperoxy Avicin D

| TEST | RESULT/REFERENCE |
|---|---|
| Appearance | White solid |
| 500 MHz ¹H NMR | Assignments and spectra - Agree with |
| Spectrum (CD₃OD) | structure |
| 500 MHz ¹³C | Assignments and spectra - Agree with |
| NMR Spectrum | structure |
| LC-MS (TIS) | m/z 2137 [M + Na]⁺ |
| Analysis | |
| High Resolution | m/z 2136.9695 [M + Na]⁺ |
| Mass Spectrum | |
| HPLC Analysis | 98.9% (area %), SunFire C18 Column, |
| | Detector @ 230 nm |
| HPLC Analysis | >99% (area %), SunFire C18 Column, ELS |
| | Detector |

Conditions for LCMS and HPLC Purity Tests

LCMS:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 34.00 | 5 | 95 |
| 34.10 | 85 | 15 |
| 39.00 | 85 | 15 |

Detection: 230 nm, MS with turbo ion spray ionization

HPLC Purity:

Column: Sunfire C18, 150 mm×4.6 mm, 3.5 μm particles

Temperature: ambient

Flow Rate: 1.0 mL/min

Solvent Gradient:

| Time | Water (%) | Acetonitrile (%) |
|---|---|---|
| (min) | (0.1% Formic Acid) | (0.1% Formic Acid) |
| 0.00 | 85 | 15 |
| 30.00 | 55 | 45 |
| 31.00 | 5 | 95 |
| 33.00 | 5 | 95 |
| 34.00 | 85 | 15 |
| 37.00 | 85 | 15 |

Detection: Photodiode array from 190 nm-370 nm (extraction at 230 nm)

ELSD, 120 ° C., 3.0 L/min nitrogen

TABLE 21
¹H and ¹³C NMR Assignments for Aglycone
region of C-11 Hydroperoxy Avicin D

| Position | ¹³C | ¹H |
|---|---|---|
| 1 | 40.8 | 1.35 m |
| | | 1.91 m |
| 2 | 27.1 | 1.66 m |
| | | 1.82 m |
| 3 | 88.4 | 3.35 m |
| 4 | 39.9 | — |
| 5 | 56.8 | 0.83 m |
| 6 | 19.4 | 1.37 m |
| | | 1.65 m |
| 7 | 34.5 | 1.32 m |
| | | 1.56 m |
| 8 | 42.5 | — |
| 9 | 51.5 | 1.77 m |
| 10 | 38.5 | — |
| 11 | 81.9 | 4.41 m |
| 12 | 123.3 | 5.68 d (4.1) |
| 13 | 149.4 | — |
| 14 | 43.7 | — |
| 15 | 36.0 | 1.53 m |
| 16 | 73.8 | 4.49 m |
| 17 | | — |
| 18 | 40.7 | 3.03 dd (4.4, 14.1 Hz) |
| 19 | 47.6 | 1.39 m |
| | | 2.49 m |
| 20 | 35.4 | — |
| 21 | 78.3 | 5.51 dd (5.6, 11.1 Hz) |
| 22 | 36.0 | 1.74 m |
| | | 2.18 dd (5.6, 13.7 Hz) |
| 23 | 28.2 | 0.98 s |
| 24 | 16.7 | 0.77 s |
| 25 | 17.6 | 1.04 s |
| 26 | 19.1 | 0.77 s |
| 27 | 26.0 | 1.51 s |
| 28 | | — |
| 29 | 29.0 | 0.89 s |
| 30 | 19.1 | 1.08 s |

TABLE 22
¹H and ¹³C NMR Assignments for C-3 Glycoside
region of C-11 Hydroperoxy Avicin D

| Position | ¹³C | ¹H |
|---|---|---|
| GlcNAc-1 | 104.3 | 4.45 d (8.2 Hz) |
| GlcNAc-2 | 57.6 | 3.62 m |
| GlcNAc-3 | 75.7 | 3.45 t (8.5 Hz) |
| GlcNAc-4 | 72.1 | 3.21 t (8.9 Hz) |
| GlcNAc-5 | 77.4 | 3.51 m |
| GlcNAc-6 | 69.6 | 3.75 m |
| | | 4.08 dd (1.5, 11.5 Hz) |
| GlcNAc—C═O | 173.1 | — |
| GlcNAc—Me | 22.8 | 1.94 s |
| Fuc-1 | 103.7 | 4.60 d (7.4 Hz) |
| Fuc-2 | 82.0 | 3.63 m |
| Fuc-3 | 74.6 | 3.62 m |
| Fuc-4 | 72.5 | 3.62 m |
| Fuc-5 | 71.4 | 3.60 m |
| Fuc-6 | 16.4 | 1.27 d (5.9 Hz) |
| Xyl-1 | 106.8 | 4.47 d (7.8 Hz) |
| Xyl-2 | 76.0 | 3.34 m |
| Xyl-3 | 77.6 | 3.37 m |
| Xyl-4 | 70.7 | 3.50 m |
| Xyl-5 | 66.8 | 3.26 m |
| | | 3.97 dd (5.2, 11.1 Hz) |

TABLE 23
¹H and ¹³C NMR Assignments for C-28 Glycoside
region of C-11 Hydroperoxy Avicin D

| Position | ¹³C | ¹H |
|---|---|---|
| Glc-1 | 95.1 | 5.33 m |
| Glc-2 | 75.9 | 3.53 m |
| Glc-3 | 78.0 | 3.36 m |
| Glc-4 | 70.9 | 3.36 m |
| Glc-5 | 78.4 | 3.31 m |
| Glc-6 | 61.9 | 3.66 m |
| | | 3.79 m |
| Rha-1 | 101.1 | 5.34 m |
| Rha-2 | 71.2 | 4.20 m |
| Rha-3 | 82.4 | 3.86 m |
| Rha-4 | 78.4 | 3.66 m |
| Rha-5 | 68.8 | 3.86 m |
| Rha-6 | 18.4 | 1.32 d (5.9 Hz) |
| Glc′-1 | 105.6 | 4.47 d (7.8 Hz) |
| Glc′-2 | 75.2 | 3.28 m |
| Glc′-3 | 78.8 | 3.52 m |
| Glc′-4 | 70.9 | 3.36 m |
| Glc′-5 | 77.5 | 3.28 m |
| Glc′-6 | 62.1 | 3.71 m |
| | | 3.82 m |
| Ara-1 | 110.7 | 5.34 m |
| Ara-2 | 83.7 | 4.09 dd (1.5, 3.7 Hz) |
| Ara-3 | 78.4 | 3.88 m |
| Ara-4 | 85.4 | 4.03 m |
| Ara-5 | 62.8 | 3.64 m |
| | | 3.37 m |

TABLE 24
¹H and ¹³C NMR Assignments for C-21 Monoterpene-Glycoside
region of C-11 Hydroperoxy Avicin

| Position | ¹³C | ¹H |
|---|---|---|
| MT₁-1 | 168.0 | — |
| MT₁-2 | 132.1 | — |
| MT₁-3 | 147.8 | 6.911 (7.8 Hz) |
| MT₁-4 | 24.1 | 2.44 m |
| MT₁-5 | 41.0 | 1.74 m |
| MT₁-6 | 80.7 | — |
| MT₁-7 | 143.8 | 5.95 dd (10.7, 17.4 Hz) |
| MT₁-8 | 115.8 | 5.22 dd (1.1, 10.0 Hz) |
| | | 5.30 dd (1.1, 17.4 Hz) |
| MT₁-9 | 56.3 | 4.33 s |
| MT₁-10 | 23.5 | 1.39 s |
| Qui-1 | 99.1 | 4.42 d (7.8 Hz) |
| Qui-2 | 75.3 | 3.27 m |
| Qui-3 | 75.4 | 3.54 t (9.3 Hz) |
| Qui-4 | 77.4 | 4.64 t (9.6 Hz) |
| Qui-5 | 70.8 | 3.49 m |
| Qui-6 | 18.0 | 1.12 d (6.3 Hz) |
| MT₂-1 | 167.6 | — |
| MT₂-2 | 132.1 | — |
| MT₂-3 | 148.2 | 6.94 t (7.8 Hz) |
| MT₂-4 | 24.2 | 2.37 m |
| MT₂-5 | 41.7 | 1.64 m |
| MT₂-6 | 73.2 | — |
| MT₂-7 | 145.5 | 5.91 dd (10.7, 17.4 Hz) |
| MT₂-8 | 112.3 | 5.05 dd (1.5, 10.7 Hz) |
| | | 5.22 dd (1.5, 17.4 Hz) |
| MT₂-9 | 56.3 | 4.32 s |
| MT₂-10 | 27.7 | 1.27 s |

### Example 4

### Biological Activity Results

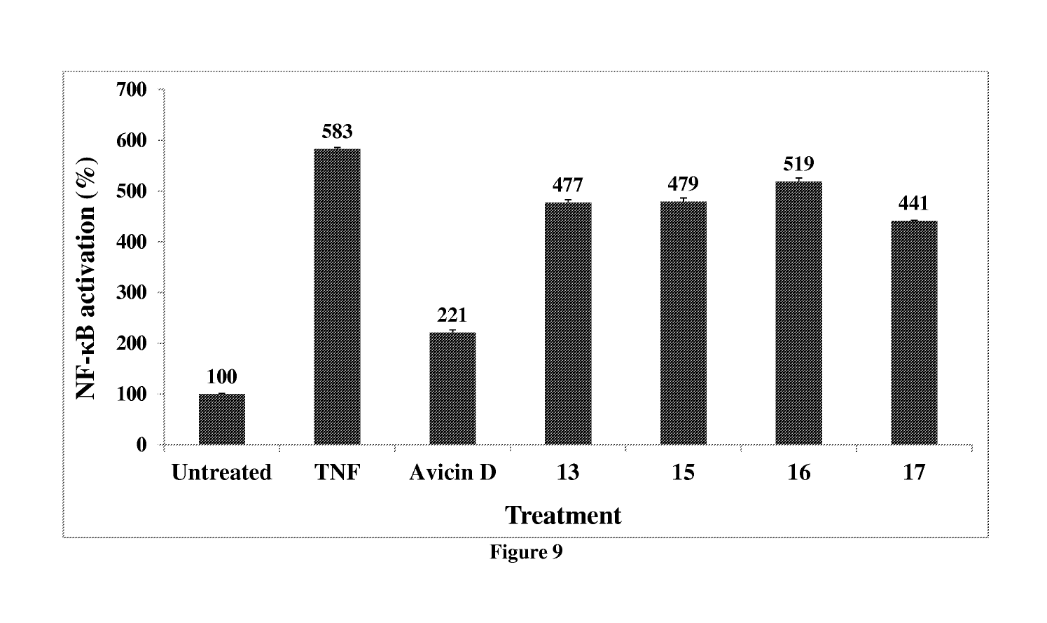

NF-κB activation was studied using p65 ab and the Trans AM NF-κB assay kit from Active motif. The results (FIG. 9) have been presented as % of untreated, which in turn was taken as 100% activation. Jurkat cells (2×10⁶/ml) were treated with avicin D or one of the derivatives (1 μM each) for 16 hrs as described in Haridas et al., Proc. Natl. Acad. Sci. (2001) 98; 11557-62, which is incorporated herein by reference. Cells were next exposed to TNF (1 nM) for 15 minutes. Table 25 correlates names, chemical formulas and codes for four of these avicin derivatives.

TABLE 25
Compound Codes and Formulas.

| Compound Reference # | Compound | Compound | Lot |
|---|---|---|---|
| in FIGS. 9, 10 and 11 | Formula | Label | reference |
| 13 | FIG. 5 | C-11 hydroxy- | ALB |
| | | Avicin D | 154491 |
| 16 | FIG. 6 | Oxidatively | ALB |
| | | rearranged | 153752-2 |
| | | Avicin D | |
| 17 | FIG. 7 | C-11 keto | ALB |
| | | Avicin D | 153384 |
| 15 | FIG. 8 | C-11 hydroperoxy | ALB |
| | | Avicin D | 154737 |

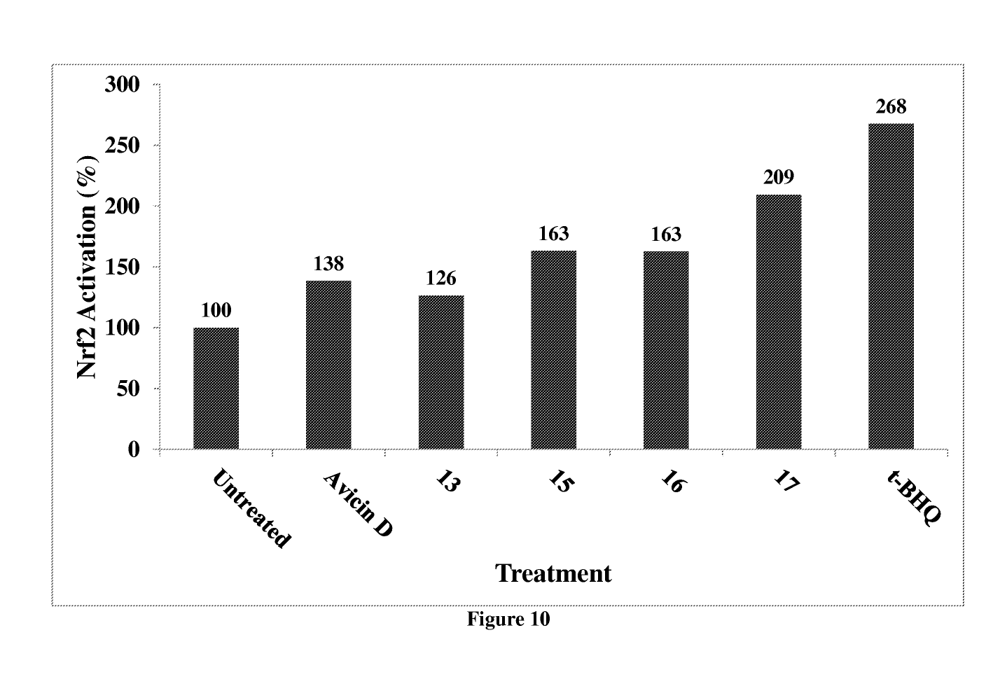

Activation of Nrf2 in response to treatment with some of the compounds from the present invention was also measured. Avicin D and t-BHQ were used as positive controls. HepG2 (hepatocarinoma) cells were treated with 1 μM of each of the compounds. Nrf2 protein was detected in the cell lysates using western blot analysis. Intensities of protein bands were quantitated using NIH Image and these results are provided in FIG. 10.

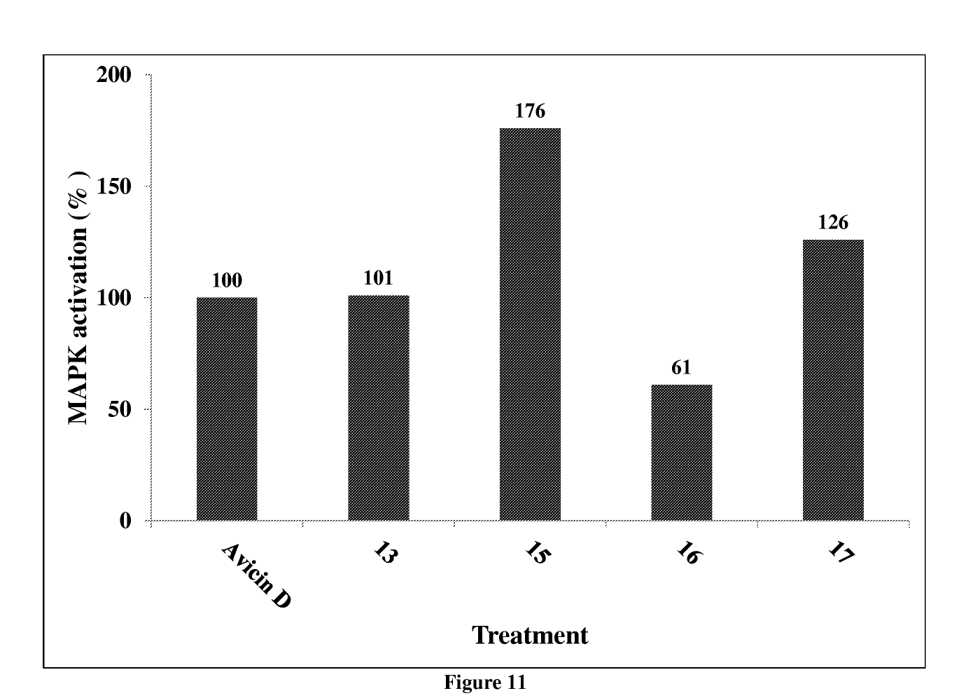

Activation of MAP kinase in response to treatment with some of the compounds from the present invention was also studied. The ability of avicin D and the different analogues to activate MAPK in PC3 cells was analysed by western blot analysis, and the results are shown in FIG. 11.

### REFERENCES

The following references to the extent that they provide exemplary procedural or other details supplementary to those set forth herein, are specifically incorporated herein by reference.
Adjei and Garren, J. Pharm. Res., 7:565-569, 1990.
Cortesi et al., Biomaterials, 19:1641-1649, 1998.
Crommelin and Midha, Medpharm Scientific Publishers, Stuttgart, 95-115, 1992.
French et al., J. Aerosol Sci., 27:769-783, 1996.
Ganderton, J. Biopharmaceutical Sciences, 3:101-105, 1992.
Gonda, In: Critical Reviews in Therapeutic Drug Carrier Systems, 6:273-313, 1990.
Gonda, In: Physico-Chemical Principles in Aerosol Delivery, Topics in Pharmaceutical Sciences, 1991.
Handbook of Pharmaceutical Excipients, 4^{th} Ed., Pharmaceutical Press, 2003.
Handbook of Pharmaceutical Salts: Properties, and Use, Stahl and Wermuth (Eds.), Verlag Helvetica Chimica Acta, 2002.
Larock, Comprehensive Organic Transformations, 2^{nd} Ed., Wiley-VCH, 1999.
Lin et al., Proc. Am. Soc. Clin. Oncol., 23:269, 2005.
Patton and Platz, Adv. Drug Del. Rev., 8:179-196, 1992.
Pharmaceutical dosage form tablets, Liberman et. al. (Eds.), NY, Marcel Dekker, Inc., 1989.
Pharmaceutical dosage forms and drug delivery systems, 6^{th} Ed., Ansel et al. (Eds.), Media, P A, Williams and Wilkins, 1995.
Remington—The science and practice of pharmacy, 20^{th} Ed., Jennaro et. al. (Eds.), Phila, Lippencott, Williams, and Wilkens, 2000.
Remington's Pharmaceutical Sciences, 16^{th} Ed., Osol (Ed.), 1445, 1980.
Rudt and Muller, J. Controlled Release, 22:263-272, 1992.
Tabata and Ikada, J. Biomed. Mater. Res., 22:837-858, 1988.
U.S. Pat. No. 6,444,233
Visser, Powder Technology, 58:1-10, 1989.
Xu et al., Curr. Biol., 13(9):790-795, 2003.
Zanen and Lamm, Int. J. Pharm., 114:111-115, 1995.

1. A compound of the formula: 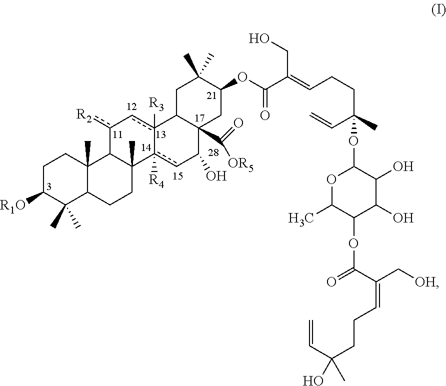
wherein:
R₁ is:
hydrogen, a monosaccharide group, a disaccharide group, an oligosaccharide group or a terpenoid group, or
alkyl_{(C≦30)}, alkenyl_{(C≦30)}, alkynyl_{(C≦30)}, aryl_{(C≦30)}, aralkyl_{(C≦30)}, acyl_{(C≦30)}, or a substituted version of any of these groups;
R₂ is hydroxy, peroxy or oxo;
R₃ is absent or methyl, provided that R₃ is absent when the atom to which is bound is part of double bond, further provided that when R₃ is absent the atom to which it is bound is part of a double bond;
R₄ is absent or methyl, provided that R₄ is absent when the atom to which is bound is part of double bond, further provided that when R₄ is absent the atom to which it is bound is part of a double bond; and
R₅ is:
hydrogen, a monosaccharide group, a disaccharide group, an oligosaccharide group or a terpenoid group, or
alkyl_{(C≦30)}, alkenyl_{(C≦30)}, alkynyl_{(C≦30)}, aryl_{(C≦30)}, aralkyl_{(C≦30)}, acyl_{(C≦30)}, or a substituted version of any of these groups;
or a pharmaceutically acceptable salt, acetal, ketal or tautomer thereof.

2. The compound of claim 1, wherein R₁ is a monosaccharide group, a disaccharide group, or an oligosaccharide group.

3. The compound of claim 2, wherein R₁ is a trisaccharide group.

4. The compound of claim 3, wherein R₁ is:

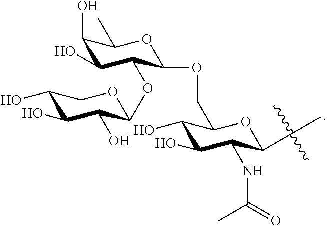

5. The compound of of claim 1, wherein R₂ is hydroxy.

6. The compound of of claim 1, wherein R₂ is peroxy.

7. The compound of of claim 1, wherein R₂ is

8. The compound of of claim 1, wherein R₃ is methyl.

9. The compound of of claim 1, wherein R₃ is absent.

10. The compound of of claim 1, wherein R₄ is methyl.

11. The compound of of claim 1, wherein R₄ is absent.

12. The compound of of claim 1, wherein R₅ is a monosaccharide group, a disaccharide group, or an oligosaccharide group.

13. The compound of claim 12, wherein R₅ is an oligosaccharide group.

14. The compound of claim 13, wherein R₅ is:

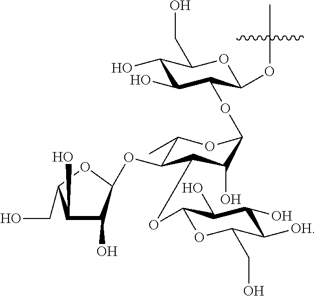

15. The compound of claim 1, further defined as:

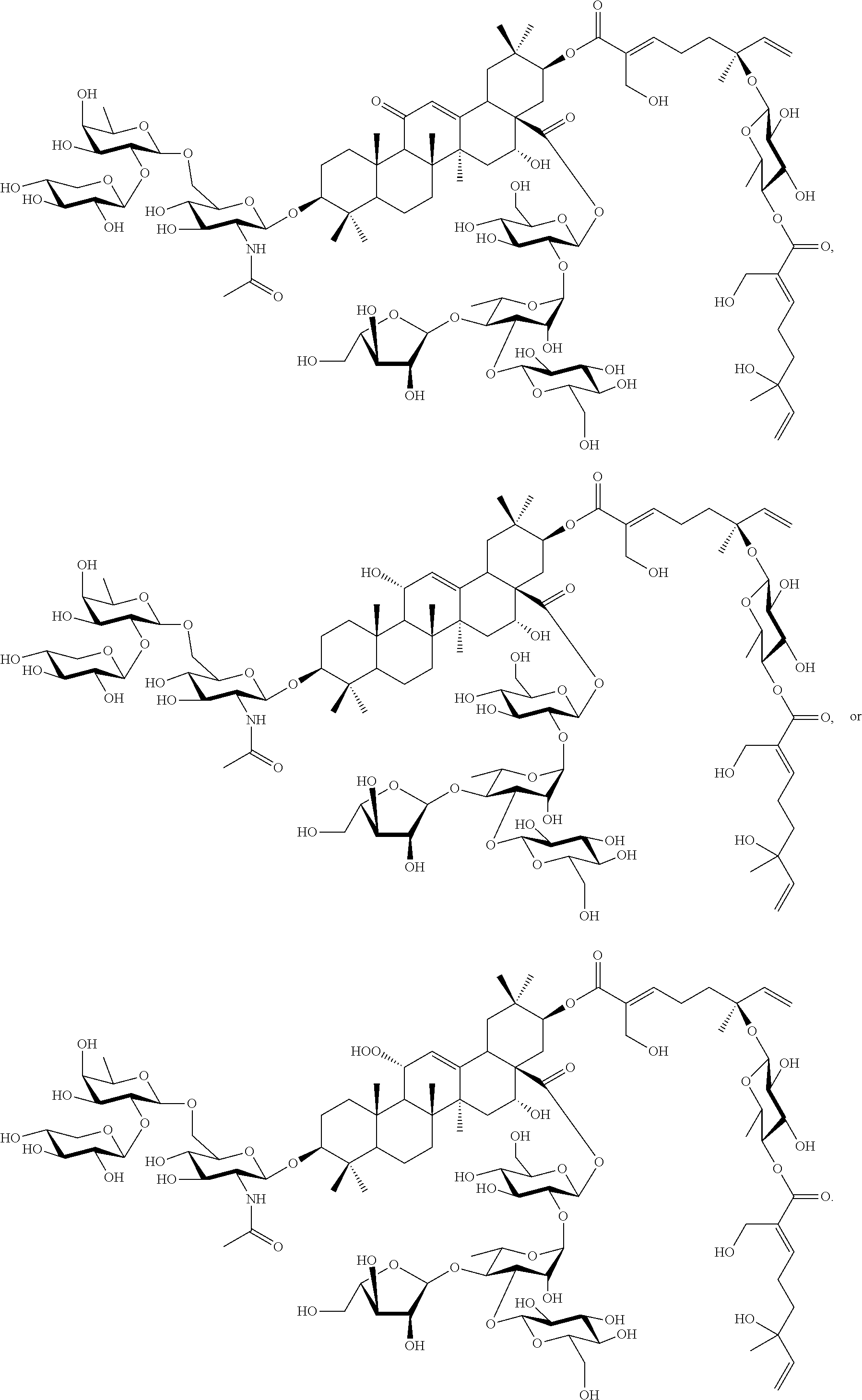

16. A compound of the formula:

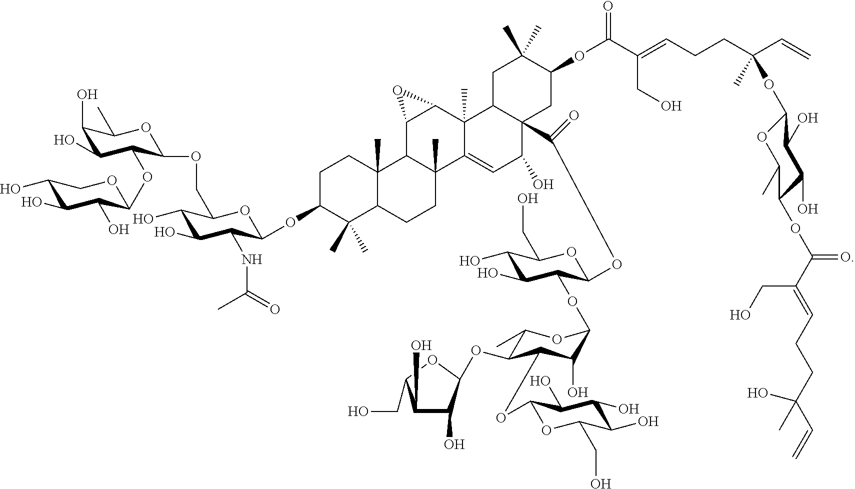

17. A pharmaceutical composition comprising the compound of claim 1 and a pharmaceutically acceptable carrier.

18. The pharmaceutical composition of claim 17, wherein the composition is formulated for oral administration.

19. The pharmaceutical composition of claim 17, further comprising one or more pharmaceutically acceptable excipients.

20. The pharmaceutical composition of claim 17, wherein the composition is formulated for controlled release.

21. A method of treating a proliferative disorder, the method comprising administering to a patient in need thereof an effective amount of a compound of of claim 1.

22. The method of claim 21, wherein the proliferative disorder is cancer.

23. A method of treating an inflammatory disorder, the method comprising administering to a patient in need thereof an effective amount of a compound of of claim 1.

24. A method of treating a disease or disorder associated with chemotaxis, the method comprising administering to a patient in need thereof an effective amount of a compound of of claim 1.

25. The method of claim 24, wherein the disease or disorder is selected from the group consisting of autoimmune diseases and irritable bowel syndrome.

26. A method of treating a metabolic disorder, the method comprising administering to a patient in need thereof an effective amount of a compound of of claim 1.

27. The method of claim 26, wherein the metabolic disorder is obesity, diabetes, and combinations thereof.

28. A kit comprising one or more compounds of of claim 1.

29. A method of synthesizing a compound of claim 1 comprising reacting Avicin D with one or more reagents to form a compound of of claim 1.
